(19) United States
(12) Patent Application Publication
(10) Pub. No.: US 2017/0001184 A1
(43) Pub. Date: Jan. 5, 2017

(54) ALKANE METATHESIS CATALYST, METHODS OF USE AND THE PREPARATION THEREOF

(71) Applicant: KING ABDULLAH UNIVERSITY OF SCIENCE AND TECHNOLOGY, Thuwal (SA)

(72) Inventors: Jean-Marie Maurince Basset, Thuwal (SA); Emmanuel Callens, Thuwal (SA); Nassima Riache, Thuwal (SA); Manoja Kumar Samantaray, Thuwal (SA); Edy Abou-Hamad, Thuwal (SA)

(73) Assignee: King Abdullah University of Science and Technology, Thuwal (SA)

(21) Appl. No.: 15/100,237

(22) PCT Filed: Nov. 26, 2014
(86) PCT No.: PCT/IB2014/003060

§ 371 Date: May 27, 2016

Related U.S. Application Data
(60) Provisional application No. 61/910,092, filed on Nov. 28, 2013.

(51) Int. Cl. B01J 31/12; B01J 31/26; C07C 6/10

(52) U.S. Cl. CPC B01J 31/122; C07C 6/10; B01J 31/26; C07C 2531/12; C07C 2531/26

(57) ABSTRACT

The invention concerns an alkane metathesis catalyst, its production and use. The catalyst comprises a Group V, VI or VII metal alkyl with the metal in its highest oxidation state, preferably Ta or W, and the alkyl of C1-C4, preferably together with alkylidene and/or alkylidyne ligands, in particular -Me, ═CH2 and ≡CH, on a metal oxide support, preferably silica partially dehydroxylated at 200 or 700° C. Substrates include cycloalkanes, preferably cyclooctane.

### PRIORITY CLAIM

This application claims priority from U.S. Provisional Patent Application No. 61/910,092, filed Nov. 28, 2013, which is incorporated by reference in its entirety.

### TECHNICAL FIELD

The invention relates to metathesis catalysts and methods of using metathesis catalysts.

### BACKGROUND

Metathesis reaction involves the exchange of bonds between the two reacting chemical species. Transformation of linear alkanes into their lower and higher homologues via alkane metathesis is an important process in the petrochemical industry. The process is often catalyzed by metal-containing compounds or complexes.

### SUMMARY

In one aspect, a catalyst can include an oxide support and a supported metal alkyl species bound to the oxide support, wherein the supported metal alkyl species can be a group V, VI or VII metal in its highest oxidation state and the alkyl group can be a C1-C4 alkyl. The oxide support can includes an oxide of silicon, an oxide of titanium, or an oxide of aluminum.

In certain embodiments,

| | |
|---|---|
| alkane | alkane |
| C—H activation | C—H activation |
| (+++) | (+++) |
| ≡M⁺OM(R)ₓ | ≡M⁺OM(H)ₓ |
| M = Ta, W, Mo, Zr, Re | M = Ta, W, Mo, Zr, Re |
| Group IV, V, VI and VII | Group IV, V, VI and VII |
| M⁺ = Si, Al, Zr, Ti, Nb (NH) | M⁺ = Si, Al, Zr, Ti, Nb (NH) |
| R = alkyl/alkylidene/yne, | |
| alkyl/alkylidyne, alkyl/bis-alkylidene | |

In certain embodiments, a supported metal alkyl species bound to the oxide support can include a moiety having a formula of (≡M′-O)ₓM(R₁)_{y}(R₂)_{z}, wherein R₁ is a C1-C4 alkylidene group or a C1-C4 alkylidyne group, wherein R₂ is a halogen or C1-C4 alkyl group or C1-C4 alkylidene, wherein x is 1, 2 or 3, y is 0 or 1, and z is 1, 2, 3, 4 or 5, and wherein M is a group IV, V, VI or VII metal. For example, when M is a group VI metal, x+2y+z is 6 when R₁ is a C1-C4 alkylidene group or each of two R₁ groups is a C1-C4 alkylidene group, and x+3y+z is 6 when R₁ is a C1-C4 alkylidyne group. “≡M′-O” can be a surface Si—O, Al—O, Zr—O, Ti—O, or Nb—O or —NH₂ group in place of —O. The support can have an oxide moiety on the surface of the support. The metal can include tungsten, molybdenum, tantalum, zirconium, rhenium or vanadium. In each case, x, y and z maintain the d⁰ oxidation state of M.

In certain embodiments, the supported metal alkyl species bound to the oxide support can include a moiety having a formula of (≡M′-O)ₓM(R₁)_{y}(R₂)_{z}, wherein ≡M′-O can be a surface Si—O or Al—O group, wherein R₁ can be a C1-C4 alkylidene group or a C1-C4 alkylidyne group, wherein R₂ can be a halogen, dialkylamide or C1-C4 alkyl group, wherein x can be 1, 2 or 3, y can be 0 or 1, and z can be 1, 2, 3, 4 or 5, and wherein M can be a group VI metal, such that x+2y+z is 6 when R₁ is a C1-C4 alkylidene group or or each of two R₁ groups is a C1-C4 alkylidene group and that x+3y+z is 6 when R₁ is a C1-C4 alkylidyne group. The dialkyl amide can be —NRₐR_{b}, where each of Rₐ or R_{b} is a C1-C6 alkyl group or an aryl group.

In certain embodiments, M can be tungsten or molybdenum. R₁ can be methylidyne. R₂ can be methyl. When x is 1, y can be 0, or y can be 1. When x is 2, y can be 1.

In certain embodiments, the supported metal alkyl species bound to the oxide support can include a moiety having a formula of (≡M′-O)ₓM(R₁)_{y}(R₂)_{z}, wherein ≡Si—O can be a surface Si—O or Al—O group, wherein R₁ can be a C1-C4 alkylidene group or a C1-C4 alkylidyne group, wherein R₂ can be a halogen, dialkylamide, or a C1-C4 alkyl group, wherein x can be 1, 2 or 3, y can be 0 or 1, and z can be 1, 2, 3, or 4, wherein M can be a group V metal, such that x+2y+z is 5 when R₁ is a C1-C4 alkylidene group or each of two R₁ groups is a C1-C4 alkylidene group and x+3y+z is 5 when R₁ is a C1-C4 alkylidyne group. The dialkyl amide can be —NRₐR_{b}, where each of Rₐ or R_{b} is a C1-C6 alkyl group or an aryl group.

In certain embodiments, M can be tantalum, or vanadium, R₁ can be methylidyne. R₂ can be methyl. The catalyst can include both a monopodal species and a bipodal species.

In certain embodiments, the supported metal alkyl species bound to the oxide support can include a moiety having a formula of (≡M′-O)ₓM(R₁)_{y}(R₂)_{z}, wherein ≡Si—O can be a surface Si—O or Al—C group, wherein R₁ can be a C1-C4 alkylidene group or a C1-C4 alkylidyne group, wherein R₂ can be a halogen, dialkylamide, or a C1-C4 alkyl group, wherein x can be 1, 2 or 3, y can be 0 or 1, and z can be 1, 2, 3, or 4, wherein M can be a group VII metal, such that x+2y+z is 7 when R₁ is a C1-C4 alkylidene group and x+3y+z is 7 when R₁ is a C1-C4 alkylidyne group. The dialkyl amide can be —NRₐR_{b}, where each of Rₐ or R_{b} is a C1-C6 alkyl group or an aryl group. In certain embodiments, M can be rhenium.

In another aspect, a method of preparing a catalyst can include dehydroxylating a first material that includes an oxide in a heated environment and grafting the dehydroxylated first material with a second material that includes a moiety having a formula of MRₓ in an inert atmosphere, wherein M can be a group V or a group VI metal in its highest oxidation state, R can be a C1-C4 alkyl group, and x can be an integer.

In certain embodiments, the first material can include an oxide of silicon, an oxide of aluminium, a mixed silica-alumina or an aminated oxide of silicon (Si—NH₂). M can be tungsten, molybdenum, tantalum, or vanadium. R can be methyl. The inert atmosphere can include argon.

In another aspect, a method of converting alkanes into higher and lower homologues can include contacting a lower alkane or higher alkane (or mixtures thereof) with a catalyst comprising an oxide support and a supported metal alkyl species bound to the oxide support, wherein the supported metal alkyl species can be a group V or a group VI metal in its highest oxidation state and the alkyl group can be a C1-C4 alkyl. The homologues are products that contain carbon chain lengths that are additive of the reactants. In other words, the products are metathesis products. Higher means compounds that contain 8 carbons or greater, for example, C8-C40 compounds. Lower means compounds that contain fewer than 8 carbons, for example, C1-C7. The alkane can be a cycloalkane, for example, a C4-C40 cycloalkane (cyclic C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, or C₃₀ compounds), or mixtures thereof. The cycloalkane can undergo metathesis at a low temperature and in a single reaction vessel (i.e., one pot). The metathesis products can be macrocycles, for example, hydrocarbon macrocycles having ring sizes of 12 to 40 carbons. The method can include separating the higher and lower homologues into a single compound. In certain embodiments, the method includes halogenating the higher and lower homologues.

In certain embodiments, the supported metal alkyl species bound to the oxide support can include a moiety having a formula of (≡Si—O)ₓM(R₁)_{y}(R₂)_{z}, wherein ≡Si—O can be a surface Si—O group, wherein R₁ can be a C1-C4 alkylidene group or a C1-C4 alkylidyne group, wherein R₂ can be a C1-C4 alkyl group, wherein x can be 1, 2 or 3, y can be 0 or 1, and z can be 1, 2, 3, 4 or S, wherein M can be a group VI metal, such that x+2y+z is 6 when R₁ is a C1-C4 alkylidene group and that x+3y+z is 6 when R₁ is a C1-C4 alkylidyne group. In certain circumstances, R₁ and R₂ can be a C1-C4 alkylidene group.

In certain embodiments, M can be tungsten, or molybdenum. R₁ can be methylidyne. R₂ can be methyl. When x is 1, y can be 0, or y is 1. When x is 2, y can be 1.

In certain embodiments, the supported metal alkyl species bound to the oxide support can include a moiety having a formula of (≡Si—O)ₓM(R₁)_{y}(R₂)_{z}, wherein ≡Si—O can be a surface Si—O group, wherein R₁ can be a C1-C4 alkylidene group or a C1-C4 alkylidyne group, wherein R₂ can be a C1-C4 alkyl group, wherein x can be 1, 2 or 3, y can be 0 or 1, and z can be 1, 2, 3, or 4, and wherein M can be a group V metal, such that x+2y+z is 5 when R1 is a C1-C4 alkylidene group and that x+3y+z is 5 when R₁ is a C1-C4 alkylidyne group.

In certain embodiments, M can be tantalum, or vanadium. R1 can be methylidyne. R₂ can be methyl.

Other aspects, embodiments, and features will be apparent from the following description, the drawings, and the claims.

### BRIEF DESCRIPTION OF THE DRAWINGS

FIG. 1(A) shows one-dimensional (1D) ¹H MAS solid-state NMR spectrum of 2 acquired at 600 MHz (14.1 T) with a 22 kHz MAS frequency, a repetition delay of 5 s, and 8 scans. FIG. 1(B) shows two-dimensional (2D) ¹H-¹H double-quantum (DQ)/single-quantum (SQ) and FIG. 1(C) shows ¹H-¹H triple-quantum (TQ)/SQ NMR spectra of 2 (both acquired with 32 scans per t₁ increment, 5 s repetition delay, 32 individual t₁ increments). FIG. 1(D) shows ¹³C CP/MAS NMR spectrum of 2 (acquired at 9.4 T (v₀(¹H)=400 MHz) with a 10 kHz MAS frequency, 1000 scans, a 4 s repetition delay, and a 2 ms contact time. Exponential line broadening of 80 Hz was applied prior to Fourier transformation. (E) 2D ¹H-¹³C CP/MAS dipolar HETCOR spectrum of 2 (acquired at 9.4 T with an 8.5 kHz MAS frequency, 2000 scans per t₁ increment, a 4 s repetition delay, 64 individual t₁ increments and a 0.2 ms contact time).

FIG. 2 shows ¹H NMR spectrum of WMe₆ in CD₂Cl₂ at 203 K.

FIG. 3 shows solution ¹³C NMR spectrum of WMe₆ in CD₂Cl₂ at 203 K.

FIG. 4 shows 2D solution ¹H-¹³C Heteronuclear Single Quantum Correlation (HSQC) NMR spectrum of WMe₆ in CD₂Cl₂ at 203 K.

FIG. 5 shows FT-IR spectroscopy of aerosol silica partially dehydroxylated at 700° C. (red curve) and WMe₆ grafted on silica (700) (green curve).

FIG. 6 shows FT-IR spectroscopy of a mixture of monopodal and bipodal (≡Si—O)ₓW≡CH(CH₃)_{y}.

FIG. 7 shows ¹³C CP/MAS NMR spectra of both ¹³C labeled (95% ¹³C) (a) WMe₆ grafted on silica-200° C. (3) and (b) WMe₆ grafted on silica-700° C.

FIG. 8 shows ¹H spin echo MAS solid-state NMR spectra of the thermal transformation of 2 (acquired on a 600 MHz NMR spectrometer under 20 KHz MAS spinning frequency, number of scans=8, repetition delay=5 s). The true sample temperatures were calibrated by separately measuring the ⁷⁹Br isotropic chemical shifts and longitudinal relaxation times of KBr.

FIG. 9 shows (A) 1D ¹H spin-echo MAS solid-state NMR spectrum of [(≡SiO)ₓW(≡CH)Me_{y}] after maintaining the temperature of 2 at 345 K for 12 hours (acquired on a 600 MHz NMR spectrometer under a 20 kHz MAS spinning frequency, number of scans=8, repetition delay=5 s) (B) 2D ¹H-¹H DQ and (C) ¹H-¹H TQ (acquired on a 600 MHz NMR spectrometer under 22 kHz MAS spinning frequency with a back-to-back recoupling sequence, number of scans=128, repetition delay=5 s number of t₁ increments=128, with the increment set equal to one rotor period of 45.45 μs) (D) ¹³C CP/MAS NMR spectrum (10 kHz MAS at the same field as above, number of scans=20000, repetition delay=4 s, contact time=2 ms, line broadening=80 Hz) (E) 2D CP/MAS HETCOR NMR spectrum acquired with short contact times of 0.2 ms under 8.5 kHz MAS, number of scans per increment=4000, repetition delay=4 s, number of t₁ increments=32, line broadening=80 Hz).

FIG. 10 shows ²⁹Si CP-MAS NMR spectrum of a mixture of monopodal and bipodal (≡SiO)ₓW≡CH(CH₃)_{y} acquired at 400 MHz with a 5 kHz MAS frequency of 5 kHz. The number of scans was 20 000, and the recycle delay was set to 5 s. A cross polarization time of 5 ms was used. An exponential line broadening of 100 Hz was applied prior to Fourier transform.

FIG. 11-I shows (A) ¹³C CP/MAS spectrum of species 5; (B) W methylidyne and W bismethylidene PMe₃ adducts (both acquired at 9.4 T (v₀ (¹H)=400 MHz) and a MAS frequency of 10 kHz, 10,000 scans. A 4 s repetition delay and a contact time of 2 ms. Exponential line broadening of 80 Hz was applied prior to Fourier transformation). FIG. 11-II shows the 2D ¹H-¹³C CP/MAS dipolar HETCOR spectrum of W methylidyne and W bis-methylidene PMe₃

adducts (acquired at 9.4 T and a MAS frequency of 8.5 kHz, 4000 scans per t₁ increment, a 4 s repetition delay, 32 individual increments and a contact time of 0.2 ms).

FIG. 12 shows (A) ³¹P CP/MAS spectrum of W methylidyne and W bis-methylidene PMe3 adducts (acquired at 9.4 T (v₀ (¹H)=400 MHz) and a MAS frequency of 10 kHz, 500 scans, 4 s repetition delay and a contact time of 2 ms. Exponential line broadening of 20 Hz was applied prior to Fourier transformation). (B) 2D ³¹P-³¹P spin-diffusion with DARR (dipolar-assisted rotational resonance) obtained with a mixing time tmix=40 ms. Sequence begins with CP using a ramped pulse on the ³¹P channel (acquired at 9.4 T and a MAS frequency of 10 kHz, 400 scans per t₁ increment, a 4 s repetition delay, 128 individual increments and a contact time of 4 ms) and (C) The 2D ¹H-³¹P CP/MAS dipolar HETCOR spectrum of W methylidyne and W bis-methylidene PMe₃ adducts (acquired at 9.4 T and a MAS frequency of 10 kHz, 400 scans per t1 increment, a 4 s repetition delay, 32 individual increments and a contact time of 1 ms).

FIG. 13 shows projection in the w2 dimension of the 2D ¹³C—¹³C double-quantum DQ/single quantum (SQ) with cross polarization for weak dipole-dipole couplings, compensated for pulse imperfection of W methylidyne and W bis-methylidene PMe₃ adducts (acquired at 9.4 T and a MAS frequency of 10 kHz, 1000 scans per t₁ increment, a 4 s repetition delay, 256 individual increments and a contact time of 3 ms).

FIG. 14A shows a graph depicting products distribution of cyclooctane metathesis catalyzed by species 2 and 5. Reaction conditions: a batch reactor, 2 and 5, cyclooctane (0.5 mL, 3.7 mmol), and 150° C. FIG. 14B shows GC chromatogram of cyclooctane metathesis products catalyzed by 1. Reaction conditions: batch reactor, 1 (300 mg, 23 μmol, W loading: 1.4% wt), cyclooctane (2 mL, 14.88 mmol), 190 h, 150° C. Conversion=70%, TON=450. The turnover number (TON) is the number of mol of cyclooctane transformed per mole of W. FIG. 14B shows GC chromatogram of cyclooctane metathesis products catalyzed by species 2. Reaction conditions: batch reactor, 1 (300 mg, 23 μmol, W loading: 1.4% wt), cyclooctane (2 mL, 14.88 mmol), 190 h, 150° C. Conversion=70%, TON=450. The turnover number (TON) is the number of mol of cyclooctane transformed per mole of W.

FIG. 15 shows a GC chromatogram of the original mixture; of the mixture after isolation of cC₁₇ and cC₂₁ and their corresponding chromatograms.

FIG. 16 shows cyclooctane metathesis catalytic performance catalyzed by species 2: TON ( [symbol] ) and conversion ( [symbol] ) of cyclooctane versus time. Reaction conditions: batch reactor, 2 (50 mg, 6.5 μmol, W loading: 2.4% wt), cyclooctane (0.5 mL, 3.7 mmol), 150° C.

FIG. 17 shows cyclooctane metathesis products selectivity catalyzed by species 2: sum of cyclic alkanes (cC₅-cC₇) ( [symbol] ) sum of macrocyclic alkanes (cC₁₂-cC₃₀) ( [symbol] ) and conversion of cyclooctane ( [symbol] ). Reaction conditions: batch reactor, 2 (50 mg, 6.5 μmol, W loading: 2.4% wt), cyclooctane (0.5 mL, 3.7 mmol), 150° C.

FIG. 18A shows a graph depicting products distribution of cyclooctane metathesis from 0.5 h to 6 h catalyzed by species 2. Reaction conditions: batch reactor, 2 (50 mg, 6.5 μmol, W loading: 2.4% wt), cyclooctane (0.5 mL, 3.7 mmol), 150° C. FIG. 18B shows a graph depicting products distribution of cyclooctane metathesis from 8 h to 80 days catalyzed by species 2.

FIG. 19 shows a schematic depicting postulated mechanism for cyclohexadecane formation from cyclooctane metathesis.

FIG. 20 shows a schematic depicting proposed mechanism for selected cyclic and macrocyclic alkanes formation from cyclooctane metathesis (ROM: Ring Opening Metathesis; RCM: Ring Closing Metathesis; Iso: double bond isomerization).

FIG. 21 shows a calibration plot of intensities versus concentration of cyclic alkanes.

FIG. 22 shows a calibration plot of cycloalkanes response factor versus carbon number.

FIG. 23 shows a ¹H NMR spectrum of the filtrate after cyclooctane metathesis typical catalytic run.

FIG. 24 shows a ¹³C NMR spectrum of the filtrate after cyclooctane metathesis typical catalytic run.

FIG. 25 shows DEPT-135 NMR of the filtrate after cyclooctane metathesis typical catalytic run.

FIG. 26 shows a plot of the log of relative retention time versus carbon number of cyclooctane metathesis reaction products in the range of C₁₆ to C₂₉ obtained by isothermal GC analysis (200° C. using a HP-5 capillary column).

FIG. 27 shows mass spectra of (a) C₁₆ alkane from cyclooctane metathesis and (b) C₁₆ octylcyclooctane

FIG. 28 shows EI spectra of cycloeicosane (C₂₀H₄₀), cyclohexadecane (C₁₆H₃₂) and cyclododecane (C₁₂H₂₄) obtained from library of GC-MS software. This figure shows the similar ion fragmentation pattern of a homologue series of macrocyclic alkanes.

FIG. 29 shows EI spectra of tetradecylcyclooctane (top) and octylcyclodecane (bottom).

FIG. 30 shows ¹H and ¹³C NMR spectra of cC₁₇.

FIG. 31 shows ¹H and ¹³C NMR spectra of cC₂₁.

FIG. 32 shows a GC-MS chromatogram cyclooctane metathesis products. Major peaks are identified to be macrocyclic alkanes. The X marks peaks indicate that the library of GC-MS software does not contain the corresponding compound.

FIG. 33 shows molar percentage of products distribution of cyclooctane metathesis from 0.5 h to 6 h (%). Reaction conditions: batch reactor, 1 (50 mg, 6.5 μmol, W loading: 2.4 wt %), cyclooctane (0.5 mL, 3.7 mmol), 150° C.

FIG. 34A shows molar products distribution of cyclooctane metathesis from 7 h to 720 h. Reaction conditions: batch reactor, 1 (50 mg, 6.5 μmol, W loading: 2.4 wt %), cyclooctane (0.5 mL, 3.7 mmol), 150° C. FIG. 34B shows products distribution of cyclooctane metathesis versus time. Mass balances for these catalytics run are initially between 30-73% (<12 h) and increase with time to 80-96%. MALDI-TOF and GPC experiments show the absence of oligomers in the filtrate (<12 h).

FIG. 35 shows a GC-chromatrogram of cyclodecane metathesis products: cyclic (in the range cC₅-cC₈) and macrocyclic alkanes (in the range cC₁₂-cC₄₀). At the end of the run, the reaction was quenched by CHCl₃.

FIG. 36 shows an expansion of GC-MS chromatogram of cyclodecane metathesis reaction. Three different alkane series are identified; cyclic alkanes, n-alkanes and n-alkyl substituted cyclohexane.

FIG. 37 shows a ¹³C NMR experiment of cyclooctane metathesis at different reaction time in a NMR Young tube (blue curve: t=0 h, red curve: t=24 h, green curve: t=72 h, pink curve: t=10 days).

FIG. 38 shows a GC-chromatogram of macrocyclic alkanes in the range cC₁₃-cC₄₀ obtained after removal of cyclic alkanes under reduced pressure of cyclooctane metathesis products.

FIG. 39A shows a GC-chromatogram of crude reaction mixture after bromination. FIG. 39B shows GC chromatogram of isolated products.

FIG. 40 shows fragmentation of cC₁₆Br.

FIG. 41 shows ¹H NMR characterization of brominated macrocyclic products.

FIG. 42 shows IR characterization of brominated macrocyclic products.

FIG. 43 shows a schematic depicting potential applications of brominated cyclooctane.

### DETAILED DESCRIPTION

Alkanes are the major constituents of petroleum. As oil reserves dwindle, the world will increasingly rely on the Fischer-Tropsch process (reductive oligomerization of CO and H2) to produce liquid hydrocarbons—specifically n-alkanes—from the vast reserves of coal, natural gas, oil shale, and tar sands, or from biomass. The energy content of U.S. coal reserves alone, for example, is about 40 times that of U.S. petroleum reserves and is comparable to that of the entire world's petroleum reserves.

Unfortunately, neither natural sources nor Fischer-Tropsch production yield alkane mixtures with a tightly controlled molecular weight (MW) distribution, which is important for varied applications. For example, n alkanes in the range of C9 to C20 constitute the ideal fuel for a diesel engine; the absence of aromatic impurities results in cleaner burning than that of conventional diesel fuel or even gasoline. n-Alkanes lower than C9, however, suffer from high volatility and lower ignition quality (cetane number). In addition to F-T product mixtures, low-carbon number, low-MW alkanes are also major constituents of a variety of refinery and petrochemical streams.

In general, there is currently no practical method for the interconversion of alkanes to give products of higher MW; this challenge provides extremely large-scale potential applications of alkane metathesis. Although hydrocracking is already a well-established process for this purpose, the formation of low-MW products from high-MW reactants (e.g., by reaction with ethane) might offer an advantage.

Any transformation of paraffin or methane to liquid paraffin is of crucial economic importance for energy (liquid fuel). Alkane metathesis represents a powerful tool for making progress in a variety of areas, perhaps most notably in the petroleum and petrochemical fields. Modern civilization is currently confronting a host of problems that relate to energy production and its effects on the environment, and judicious application of alkane metathesis to the processing of fuels such as crude oil and natural gas may well afford solutions to these difficulties.

Transformation of linear alkanes into their lower and higher homologues via alkane metathesis is an important process in the petrochemical industry. See, for example, Basset, J. M. et al., Angew. Chem., Int. Edit. 2006, 45, 6082-6085, which is incorporated by reference in its entirety. Two main families of catalytic systems have been reported for alkane metathesis: (i) a dual catalyst system which relies on a dehydrogenation/hydrogenation catalyst combined with an olefin metathesis catalyst and (ii) a “multifunctional” single site catalyst supported on various oxides which is able to achieve these three reactions. See, for example, Burnett, R. L. et al., J. Catal. 1973, 31, 55-64; Haibach, M. C. et al., Acc. Chem. Res. 2012, 45, 947-958; Basset, J. M. et al., Acc. Chem. Res. 2010, 43, 323-334, each of which is incorporated by reference in its entirety. Since the first disclosed silica-supported tantalum hydride, there have been reports about various single-site supported catalysts for alkane metathesis employing Ta and W-polyhydrides directly linked to silica, silica-alumina and alumina. See, for example, Vidal, V. et al., Science 1997, 276, 99-102; Le Roux, E. et al., Angew. Chem., Int. Edit. 2005, 44, 6755-6758: Taouftik, M. et al., J. Top. Catal. 2006, 40, 65-70, each of which is incorporated by reference in its entirety. These catalysts have been synthesised and characterised at the molecular and atomic level. Most of them were found to transform light alkanes into their lower and higher homologues. See, for example, Rascon, F. et al., J. Organomet. Chem. 2011, 696, 4121-4131, which is incorporated by reference in its entirety. In these instances, the first step of C—H bond activation occurred on the metal hydride, and the resulting alkyl species were assumed to undergo either a process of alpha or beta-H elimination to give the corresponding carbene or olefin, both of which are key intermediates for the olefin metathesis process. See, for example, Chauvin, Y. Angew. Chem., Int. Edit. 2006, 45, 3740-3747, which is incorporated by reference in its entirety. Although the most active catalysts are generated from surface metal hydrides, supported catalysts which contain a neopentyl/neopentylidene moiety can also be active in alkane metathesis. It was therefore assumed that an alkyl/hydride functional group is needed to provide an alkylidene to convert alkenes intermediates via a metallacyclobutane. See, for example, Copéret, C. Chem. Rev. 2010, 110, 656-680; Blanc, F. et al., P. Natl. Acad. Sci. USA 2008, 105, 12123-12127, each of which is incorporated by reference in its entirety.

Alkane metathesis and the interaction between oxide supports and organometallic complexes were studied in the field of surface organometallic chemistry (SOMC). Alumina supported tungsten hydride, W(H)₃/Al₂O₃, can catalyze alkane metathesis. The derivative supported tungsten hydrides highly unsaturated are electron-deficient species that are very reactive toward the C—H and C—C bonds of alkanes. See, for example, Szeto, K. C. et al., Catal Sci Technol 2012, 2, 1336-1339, which is incorporated by reference in its entirety. They show a great versatility in various other reactions, such as cross-metathesis between methane and alkanes, cross-metathesis between toluene and ethane, or even methane non-oxidative coupling. See, for example, Szeto, K. C. et al., Chem Commun 2010, 46, 3985-3987, which is incorporated by reference in its entirety. Moreover, tungsten hydride exhibits a specific ability in the transformation of iso-butane into 2,3-dimethylbutane as well as in the metathesis of olefins or the selective transformation of ethylene into propylene. See, for example, Mazoyer, E. et al., Acs Catal 2011, 1, 1643-1646; Mazoyer, E. et al., Chem Commun 2012, 48, 3611-3613, each of which is incorporated by reference in its entirety.

W/Ta alkylidene complexes discovered by Wilkinson and Schrock can be active catalysts in olefin metathesis, which is one of the various steps occurring in single-site alkane metathesis. See, for example, Shortland, A. J. et al., J. Am. Chem. Soc. 1974, 96, 6796-6797; Schrock. R. R. J. Am. Chem. Soc. 1974, 96, 6796-6797, each of which is incorporated by reference in its entirety. Thus, the preparation of such species as single sites on surfaces together with alkyl/hydride is of high interest for alkane metathesis. However, in the past, several approaches to synthesize surface methylidene species have been used with little success. See, for example, Buffon, R. et al., J. Chem. Soc., Dalton Trans. 1994, 1723-1729; Le Roux, E. et al., Organometallics 2005, 24, 4274-4279, each of which is incorporated by reference in its entirety.

Previously it was reported that silica supported W-alkyl species are not effective for alkane metathesis, but as described herein, silica supported ≡Si—O—W(Me)₅ species can actually increases the activity several fold as compared to the reported silica supported W-alkyl/alkylidyne and W-hydride species. See, for example, Le Roux, E. et al., Angew Chem Int Edit 2005, 44, 6755, which is incorporated by reference in its entirety. The activity of the catalyst can be better than previously reported and patented alumina supported W-hydride catalyst.

Macrocyclic alkanes are a class of molecules with high value interest in industry. For instance, macrocyclic-alkanes and their methylated analogues are biomarkers isolated from torbanite of Botryococcus Braunil used in studies of environmental change. See. M. Audino, K. Grice, R. Alexander, C. J. Boreham, R. I. Kagi, Geochim Cosmochim Ac 2001, 65, 1995, M. Audino, K. Grice, R. Alexander, R. I. Kagi, Org Geochem 2001, 32, 759, and M. Audino, K. Grice, R. Alexander, R. Kagi, Org Geochem 2004, 35, 661, each of which is incorporated by reference I nits entirety. Macrocyclic alkanes could also serve as building blocks in the synthesis of macrolides. In fact, the carbon skeleton is found in several macrocyclic musk (e.g. muscone, civetone, exaltolide) used as olfactory molecules. See, A. Gradillas, J. Perez-Castells, Angew Chem Int Edit 2006, 45, 8086, which is incorporated by reference in its entirety. Today, a facile access to various macrocyclic alkanes size remains a synthetic challenge. The late valuable transformation which converts given linear alkanes to higher linear alkanes, namely alkane metathesis is an interesting strategic tool. See, J. M. Basset, C. Coperet, D. Soulivong, M. Taoufik, J. Thivolle-Cazat, Angew. Chem. Int. Edit. 2006, 45, 6082, which is incorporated by reference in its entirety. To date, two alkane metathesis catalytic systems have been reported. See, J. M. Basset, C. Coperet, D. Soulivong, M. Taoufik, J. T. Cazat, Accounts Chem Res 2010, 43, 323 and M. C. Haibach. S. Kundu, M. Brookhart, A. S. Goldman, Accounts Chem Res 2012, 45, 947, each of which is incorporated by reference in its entirety. The alkane metathesis via a single catalytic system was discovered in the 90's with silica supported tantalum hydride (see V. Vidal, A. Theolier, J. Thivolle-Cazat, J. M. Basset, Science 1997, 276, 99, which is incorporated by reference in its entirety) and extended to oxides supported group V hydrides later on. These systems act as multifunctional supported catalyst, which transform acyclic light alkanes into a mixture of their lower and higher homologues. See, C. Coperet, Chem Rev 2010, 110, 656, and C. Coperet, M. Chabanas, R. P. Saint-Arroman, J. M. Basset, Angew Chem Int Edit 2003, 42, 156, each of which is incorporated by reference in its entirety. Another catalytic system employs a tandem strategy with two different metals, one metal for alkane (de)hydrogenation step and another one for olefin metathesis transformation. This tandem catalytic system generally operates at high temperature until the recent development of a homogeneous iridium-based pincer complex with an olefin metathesis catalyst. See, R. L. Burnett, T. R. Hughes, J Catal 1973, 31, 55, A. S. Goldman, A. H. Roy, Z. Huang, R. Ahuja, W. Schinski, M. Brookhart, Science 2006, 312, 257, and J. Choi, A. H. R. MacArthur, M. Brookhart, A. S. Goldman. Chem Rev 2011, 111, 1761, each of which is incorporated by reference in its entirety.

A catalyst for metathesis can include an oxide or partially aminated support and a supported metal alkyl species bound to the oxide support, wherein the supported metal alkyl species is a group V or a group VI metal in its highest oxidation state and the alkyl group is a C1-C4 alkyl. For example, a metal alkyl species can include a polymethyl tungsten complex possessing no β-H, which can be a suitable alternative candidate to the neopentyl ligand to generate in situ surface W-methylidene species in its highest oxidation state.

A supported metal alkyl species bound to the oxide support can include a moiety having a formula of (≡M—O)ₓM(R₁)_{y}(R₂)_{z}, wherein R₁ is a C1-C4 alkylidene group or a C1-C4 alkylidyne group, wherein R₂ is a halogen or C1-C4 alkyl group or C1-C4 alkylidene, wherein x is 1, 2 or 3, y is 0 or 1, and z is 1, 2, 3, 4 or 5, and wherein M is a group VI metal, such that x+2y+z is 6 when R₁ is a C1-C4 alkylidene group and that x+3y+z is 6 when R₁ is a C1-C4 alkylidyne group. “≡M-O” can be a surface Si—O, Al—O and Si—NH₂ group. The oxide support can have an oxide moiety on the surface of the support. The metal can include tungsten, molybdenum, tantalum, rhenium or vanadium. In certain embodiments. R₁ or R₂ can be a hydride.

A supported metal alkyl species bound to the oxide support can include a moiety having a formula of (≡Si—O)ₓM(R₁)_{y}(R₂)_{z}, wherein ≡Si—O is a surface Si—O group, wherein R₁ is a C1-C4 alkylidene group or a C1-C4 alkylidyne group, wherein R₂ is a C1-C4 alkyl group wherein x is 1, 2 or 3, y is 0 or 1, and x is 1, 2, 3, or 4, wherein M is a group V metal, such that x+2y+z is 5 when R1 is a C1-C4 alkylidene group and x+3y+z is 5 when R₁ is a C1-C4 alkylidyne group.

A method of converting alkanes into higher and lower homologues can include contacting lower alkanes or higher alkanes with a catalyst comprising an oxide support and a supported metal alkyl species bound to the oxide support, wherein the supported metal alkyl species is a group V or a group VI metal in its highest oxidation state and the alkyl group is a C1-C4 alkyl.

The C1-C4 alkyl group can be a methyl group, an ethyl group, a propyl group or a butyl group. Preferably, the C1-C4 group is not branched.

The oxide support binds the metal via a surface oxo bond. The oxide support can be a silicon oxide, an aluminum oxide, a titanium oxide, a tungsten oxide, a molybdenum oxide, a tantalum oxide, or other compatible oxide such as partially aminated surface oxide. The oxide support can be treated to remove surface water or hydroxyl content, for example through heating.

Given that the most active supported catalysts for single-site alkane metathesis are d⁰ W(VI) complexes, a well-defined homoleptic hexamethyltungsten complex can be immobilized to assess if its transformation into a W-methylidene can affect the catalytic performance of this alkane metathesis process.

WMe₆, (1) initially discovered by Wilkinson, can be used as a precursor. A well-defined supported ≡Si—O—W(Me)₅ 2 (Scheme 1) can be prepared and characterized, at the molecular level; its activity towards alkane metathesis and the isolation of a silica supported W methyl/methylidyne species can be studied.

The synthesis and full characterisation of a well-defined silica-supported ≡Si—O—W(Me)₅ species is described in the Example section. It is a stable material at moderate temperature, whereas the homoleptic parent complex decomposes above −20° C., demonstrating a stabilizing effect of immobilisation of the molecular complex. Above 70° C. the grafted complex produces two methylidyne surface complexes [(≡SiO—)W(≡CH)Me₂] and [(≡SiO—)₂W(≡CH)Me. All these silica supported complexes are highly active precursors for propane metathesis reactions.

WMe₆ can be grafted on variously dehydroxylated silica (at 200° C. and 700° C.) surfaces using surface organometallic strategies and tools. Solid-state NMR combined with computational modeling can offer support for the structure of a well-defined supported W species, ≡Si—O—WMe₅, a surface species that is much more stable than the homoleptic parent complex in solution. The grafting of this WMe₆ homoleptic species can allow the observation by solid state NMR the temperature dependence of the methyl ligand fluxionality at room temperature. Solid-state NMR can be used to qualitatively determine the podality (i.e., monopodal vs bipodal) of the grafted complex on silica. Thermal studies on ≡Si—O—WMe₅ 2 can be used isolate a supported W-methylidyne/methyl complex, which can be confirmed by experimental and theoretical studies. These complexes can be more active than the previously reported silica supported W complexes in alkane metathesis, with a TON of 127 at 150° C. for ≡Si—O—WMe₅.

### Macrocyclic Alkanes

Macrocyclic alkanes are a class of molecules with high value interest in industry. Macrocyclic alkanes can be used as building blocks in the synthesis of macrolides. However, currently there is no practical method for the interconversion of cyclic alkanes to give higher MW macrocyclic alkanes. Indeed, the entropy in the formation of macrocylic rings is a barrier for the synthesis of macrocyclic musks. Thus, the formation of large ring represents synthetic challenges. The simplest approach to build large rings would be to make a long chain with functionality at each end such that the two ends of a chain can react to close the ring through the formation of a new carbon-carbon bond. However, the entropy dictates that the likelihood of meeting of the ends of a chain is lower than that of one end of a chain reacting with an end of another chain. Repetition of this process leads to polymerization. The disclosed method has been developed to over the problem posed by the entropy and polymerization. For example, metathesis of cylcooctane or cyclodecane as starting materials allows formation of a wide range of macrocyclic alkanes with no observable polymers.

The cyclic alkane metathesis catalyzed by a multifunctional supported W single catalytic system can lead to a wide distribution of macrocyclic alkanes in the range of C₁₂ to C₄₀. The main advantage of the W single catalyst system is that W single catalyst can promote different elementary steps. The macrocyclic alkanes can also be post-functionalized with the multifunctional supported W single catalytic system towards valuable synthetic musks. Since they are new materials not all the possible applications are known yet, but their potential as a family of new cyclic alkanes is huge.

The family of new macrocyclic compounds can be prepared by a single alkane metathesis reaction:

xCₙH₂ₙ→yCₘH₂ₘ (with 5<m<7 and 12<m<40)

The existing catalytic systems have employed a tandem strategy with two different metals, one metal for alkane (de)hydrogenation and another for olefin metathesis. This tandem catalytic system generally has operated at high temperature until the recent development of the tandem use of an iridium-based pincer complex and a Schrock-type catalyst. In 2008, Goldman and Scott described a tandem catalytic system comprising an Ir-pincer catalyst associated with Mo-based metathesis catalyst for the production of cycloalkanes with specific carbon numbers. In contrast, the metathesis reaction of cyclic alkanes (e.g. cyclooctane and high homologues) can occur at moderate temperature (150° C.) using a multifunctional supported single catalytic system, i.e. a “single site catalyst” composed of a transition metal supported on various oxides which behaves as a multifunctional catalyst. While the tandem system produces 80% polymer which renders the isolation of macrocyclic compounds difficult and does not give a wide distribution of macrocylic alkanes but just a multiple carbon number of the starting material (2n, 3n, 4n, . . . ), the single site catalyst produces no polymeric products and generate a wide distribution of macrocylic alkanes from C₁₂ to C₄₀. This selectivity is ascribed to a distinct mechanism for the multifunctional catalyst leading to a steady state low concentration of free cycloalkene. Moreover, no polymeric products were observed at the end of the catalytic run. The cycloalkane metathesis products are only cyclic and macrocylic alkanes, and cyclic alkanes can easily be removed by reduced pressure leading to a mixture of purely macrocylic alkanes. Moreover, a specific macrocyclic alkane can be isolated from a mixture of macrocyclic alkanes from C₁₂ to C₄₀ using fractional gas chromatography for further functionalization.

### Examples

### Preparation and Characterization of ≡Si—O—W(Me)₅ on SiO₂₋₇₀₀

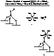

Grafting of 1 on silica has already been reported by Whan, though the system, in 1972, was poorly characterised by today's standards. See, for example, Smith, J. et al., J. Chem. Soc., Dalton Trans. 1974, 1742-1746: Mowat, W., Angew. Chem., Int. Edit. 2003, 42, 156-181, each of which is incorporated by reference in its entirety. In the following this step was re-examined using the appropriate analytical tools of modern surface organometallic chemistry (e.g., solid state NMR. IR, and elemental analysis). See, for example, Coperet, C. et al., Angew. Chem., Int. Edit. 2003, 42, 156-181, which is incorporated by reference in its entirety.

A modified synthetic protocol was employed for the synthesis of 1. See, for example, Kleinhenz, S. et al., Chem-Eur. J. 1998, 4, 1687-1691, which is incorporated by reference in its entirety. Starting from freshly sublimed WCl₆ in CH₂Cl₂, three equivalents of Me₂Zn yielded the desired complex 1 (12% yield). Solution NMR spectroscopy experiments (¹H, ¹³C and ¹H-¹³C HSQC) on the product in CD₂Cl₂ are consistent with the formation of 1, and also agree with previously reported spectroscopic data (see SI). Next, the grafting of 1 was realised by stirring a mixture of an excess of 1 and silica which had been partially dehydroxylated at 700° C. (i.e., SiO₂₋₇₀₀, which contains, 0.3±0.1 mmol silanol groups per gram) at 223 K under an inert atmosphere of argon. After several washing cycles with pentane and drying under high vacuum, the resulting yellow powder 2 contains 3.5-3.9% wt tungsten and 1.1-1.3% wt carbon as determined by elemental analysis (C/W ratio=5+/−0.1, compared to the expected value of 5).

An IR spectrum of 2 showed decreased intensity of the bands at 3742 cm⁻¹, which are associated with isolated and geminal silanols. For species 2, two new groups of bands in the 3014-2878 and 1410 cm⁻¹ regions were observed. These are assigned to ν(CH) and δ(CH) vibrations of the methyl ligands bonded to tungsten (see SI). Htydrogenolysis of 2 at 150° C. produced 5 equivalents of CH₄ per W atom. Mass balancing and gas quantification are consistent with 2 being assigned to ≡Si—O—W(Me)₅.

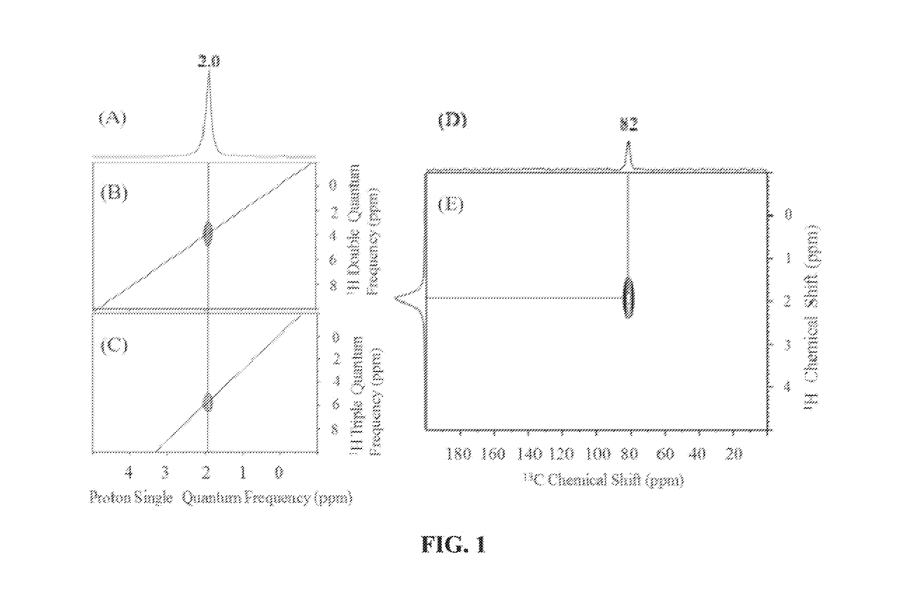

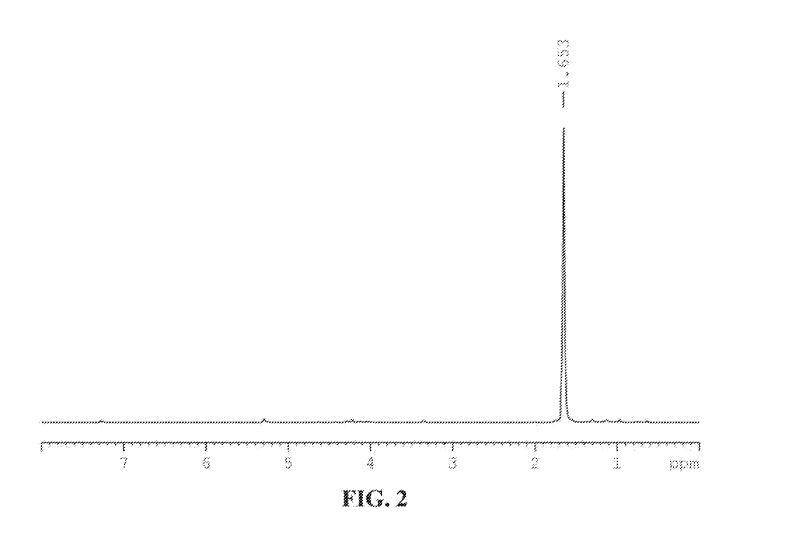

Further spectroscopic analyses of 2 were also conducted with solid-state NMR. The ¹H magic-angle spinning (MAS) solid-state NMR spectrum of 2 displays one signal at 2.0 ppm (FIG. 1A) which auto-correlates in double-quantum (DQ) and triple-quantum (TQ) NMR experiments under 22 kHz MAS as shown in FIGS. 1B and 1C respectively. See, for example, Geen, H. et al., Chem. Phys. Lett. 1994, 227, 79-86, which is incorporated by reference in its entirety. This strong autocorrelation peak is attributed to the methyl groups (2.0 ppm chemical shift in the single quantum frequency; 4.0 and 6.0 ppm in indirect dimensions of the DQ and TQ spectra, respectively). The ¹³C CP/MAS NMR spectrum shows a single peak at 82 ppm (FIG. 1D). This carbon resonance correlates with the protons at a chemical shift of 2.0 ppm, as indicated in the 2D ¹H-¹³C HETCOR NMR spectrum recorded with a contact time of 0.2 ms (FIG. 1E). The ¹H and ¹³C chemical shifts are similar to those observed in the solution NMR spectra of molecular 1. Note that grafting of 1 on oxide supports could result in the formation of monopodal or bipodal grafted species due to strained silica ring defects produced after thermal dehydroxylation. See, for example, Fleischman, S. D. et al., J. Am. Chem. Soc. 2011, 133, 4847-4855, which is incorporated by reference in its entirety.

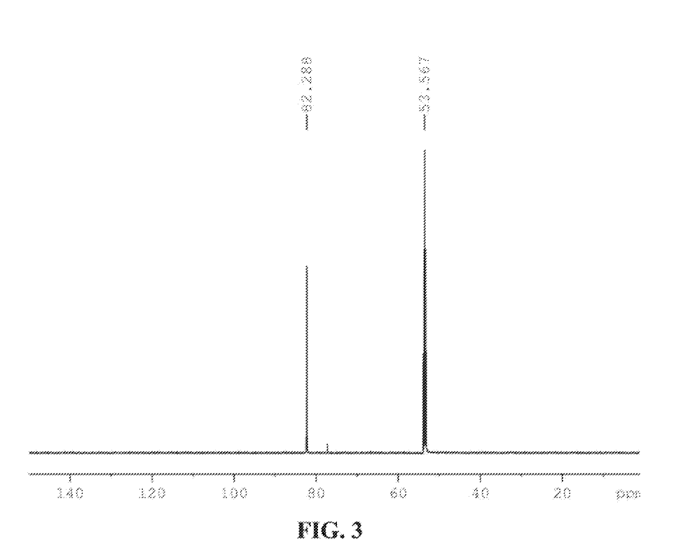

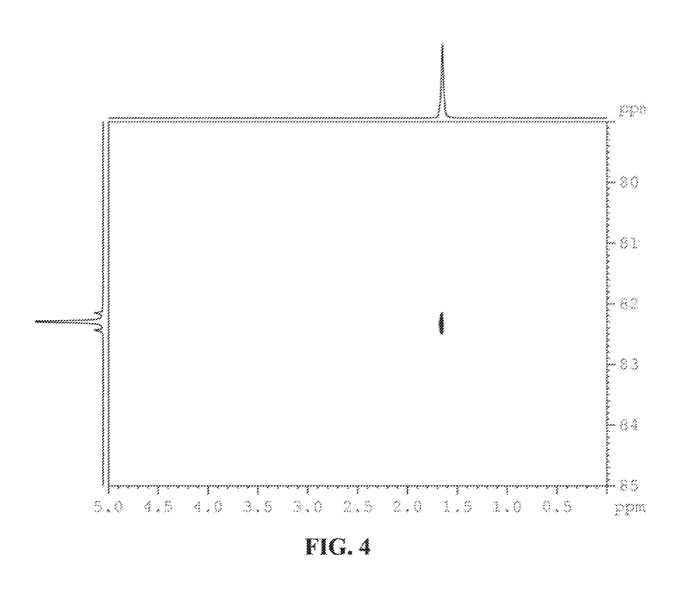

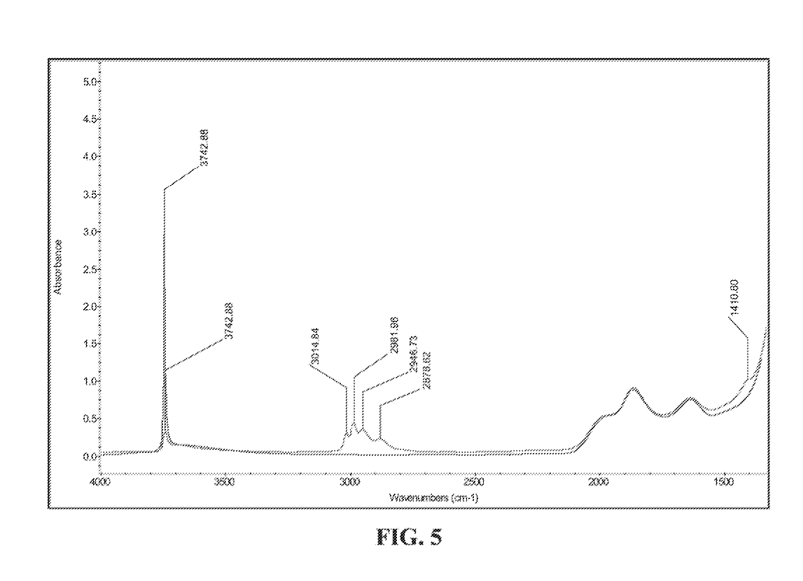

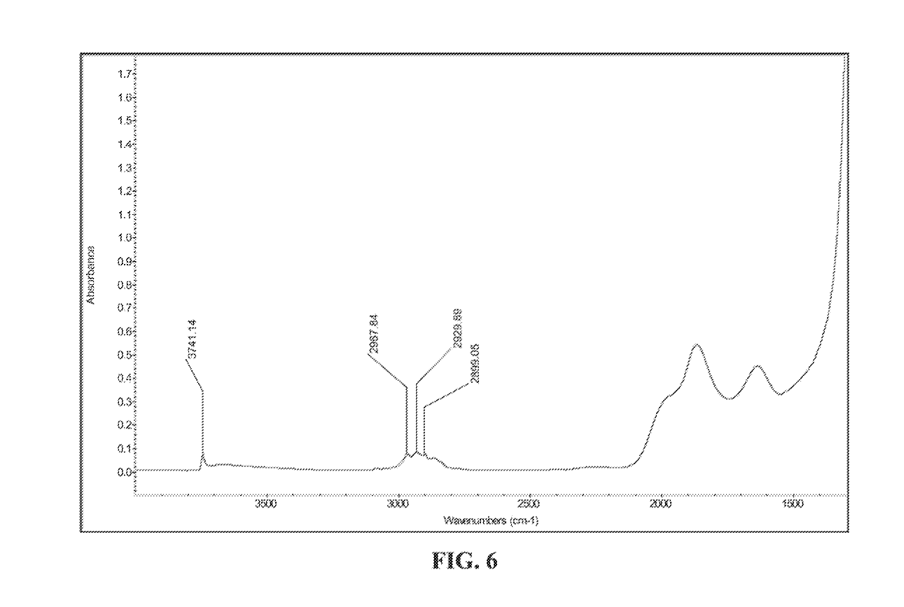

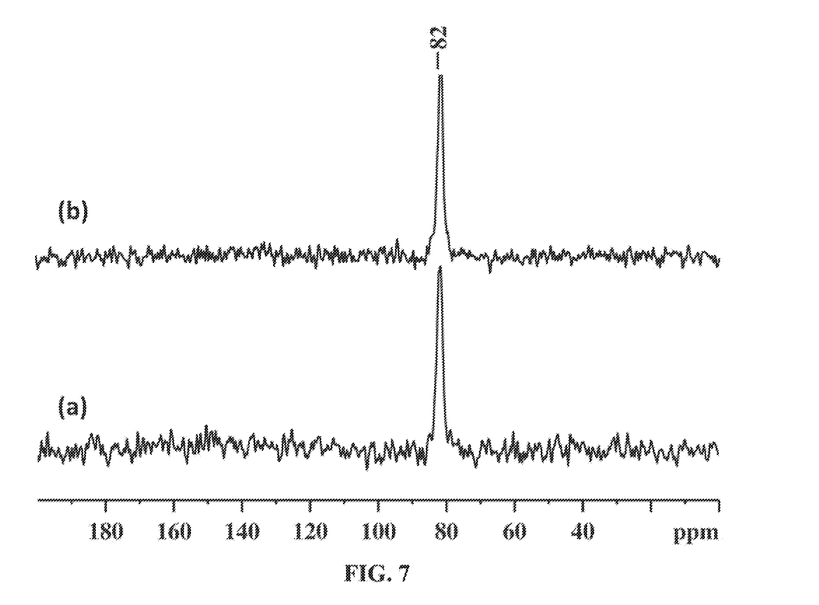

FIG. 2 shows ¹H NMR spectrum of WMe₆ in CD₂Cl₂ at 203 K. FIG. 3 shows solution ¹³C NMR spectrum of WMe₆ in CD₂Cl₂ at 203 K. FIG. 4 shows 2D solution ¹H-¹³C Heteronuclear Single Quantum Correlation (HSQC) NMR spectrum of WMe₆ in CD₂C₂ at 203 K. FIG. 5 shows FT-IR spectroscopy of aerosol silica partially dehydroxylated at 700° C. (red curve) and WMe₆ grafted on silica (700) (green curve). FIG. 6 shows FT-IR spectroscopy of a mixture of monopodal and bipodal (≡Si—O)ₓW≡CH(CH₃)_{y}. FIG. 7 shows ¹³C CP/MAS NMR spectra of both ¹³C labeled (95% ¹³C) (a) WMe₆ grafted on silica-200° C. (3) and (b) WMe₆ grafted on silica-700° C. Both spectra were acquired at 400 MHz with a 10 kHz MAS frequency, 1000 scans, a 4 s, repetition delay, and a 2 ms contact time and ambient sample temperature. An exponential line broadening of 80 Hz was applied prior to Fourier transform. ¹H and ¹³C solid-state NMR spectroscopy of a ¹³C enriched sample of 2 (95% ¹³C labelled) did not indicate the presence of signal at or near 0 ppm (in both spectra) which would indicate methyl transfer to an adjacent silicon atom of silica and hence the formation of a bipodal species [(≡Si—O)₂W(Me)₄][≡Si-Me] (see FIG. 7).

Preparation and Characterization of ≡Si—O—W(Me)₅ and (≡SiO—)₂W(Me)₄ on SiO₂₋₂₀₀

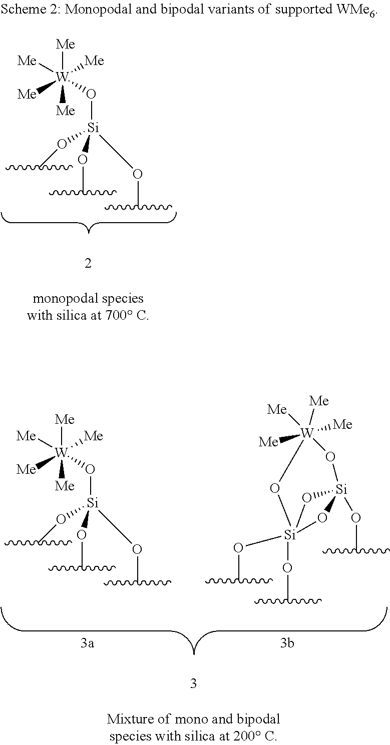

In addition, the grafting of WMe₆ was examined on silica which had been partially dehydroxylated at 200° C. (SiO₂₋₂₀₀). Immobilizing an organometallic species on less dehydroxylated silica leads frequently to a mixture of monopodal and bipodal species (Scheme 2). See, for example, Gajan, D. et al., New J. Chem. 2011, 35, 2403-2408, which is incorporated by reference in its entirety. ¹³C CP/MAS NMR spectra of 1 supported on silica treated at 200° C. (species 3) and 700° C. (species 2) both display similar chemical shifts of the methyl groups attached to the W metal at room temperature. This suggests that the monopodal species cannot be distinguished from the bipodal species of 3 at room temperature (see FIG. 7).

Evaluation of the Apparent Catalytic Activity of 2 and 3 for Propane and n-Decane Metathesis

After the synthesis and characterization of complex 2, its efficiency as a catalyst precursor for alkane metathesis reactions was investigated. Two supported catalyst systems were found to be able to convert alkanes into higher and lower homologues: i) supported metal hydrides MHₓ (M=Ta or W; x=1-3) and ii) supported M(neopentyl)ₓalkylidene/alkylidyne species (M=Ta, W or Mo; x=1-3).

Although no catalysts containing only sp³ alkyl ligands have been previously disclosed, complex 2 can be an excellent candidate for the alkane metathesis reaction. The intuitively easier loss of methane vs neopentane, via the σ-bond metathesis step, potentially offers a significant advantage when using catalyst 2 relative to a neopentyl-containing catalyst.

In previous work, the propane metathesis reaction could be the standard catalytic reaction, and thus to compare the catalytic activity of 2 with earlier results, the catalytic reaction was conducted under the same reaction conditions (a batch reactor, 1 atm of propane, and over a 5 day period at 150° C.). The experimental results confirm the hypothesis of increased catalytic activity for 2 relative to the prior species. Indeed, propane was successfully catalyzed when introducing 2 into the reaction (127 TONs) and appears to compare favourably with the previously reported inactive catalyst ≡Si—O—W(≡C/Bu)(CH₂tBu)₂ or the relatively much less reactive complex ≡Si—O—WHₓ (8 TONs). See, for example, Le Roux. E. et al., J. Adv. Synth. Catal. 2007, 349, 231-237, which is incorporated by reference in its entirety. As anticipated, when using 3 in the reaction vessel, the propane metathesis reaction was less efficient (47 TONs), and in support of the notion that the higher functional number of methyl groups on the silica surface provides better activity (see Table 1).

TABLE 1
Propane alkane metathesis: activity (TON) and alkane product selectivities of W
catalyst precursors 2 and 3 at 150° C.

| | | Product selectivity [%]^{[b]} | | | |
|---|---|---|---|---|---|
| catalyst precursors | TON (conversion)^{[a]} | Methane | Ethane | Butanes^{[c]} | Pentanes^{[d]} |
| ≡SiO₂₋₇₀₀—W(Me)₅ | 127 (12%) | 2 | 54 | 33/4 | 6/1 |
| ≡SiO₂₋₂₀₀—W(Me)ₓ | 47 (5%) | 7 | 56 | 22/2.5 | 9/2 |

^{[a]}TON is expressed in mol of propane transformed per mol of W.
^{[b]}The selectivities are defined as the amount of product over the total amount of products. Ratio of linear and branched alkanes:
^{[c]}C4/i-C4,
^{[d]}C5/i-C5.

The alkane product distribution when using these two different supported species in the reaction vessel is very similar: the major alkane products are ethane and butanes and the minor products are methane and pentanes. These products are produced since a [2+2] cycloaddition of propene with W-alkylidenes would yield two different W-metallacyclobutanes as intermediates. The steric interactions between positions [1,2] and [1,3] of the substituents on the W-metallacyclobutanes direct the alkene selectivity which upon hydrogenolysis yields the observed alkanes (See Scheme 3). See, for example, Le Roux, E. et al., J. Am. Chem. Soc. 2014, 126, 13391-13399, which is incorporated by reference in its entirety. The formation of branched alkanes results from the competitive a bond activation of CH₂ versus CH₃ groups of the propane, which is well-documented in the literature.

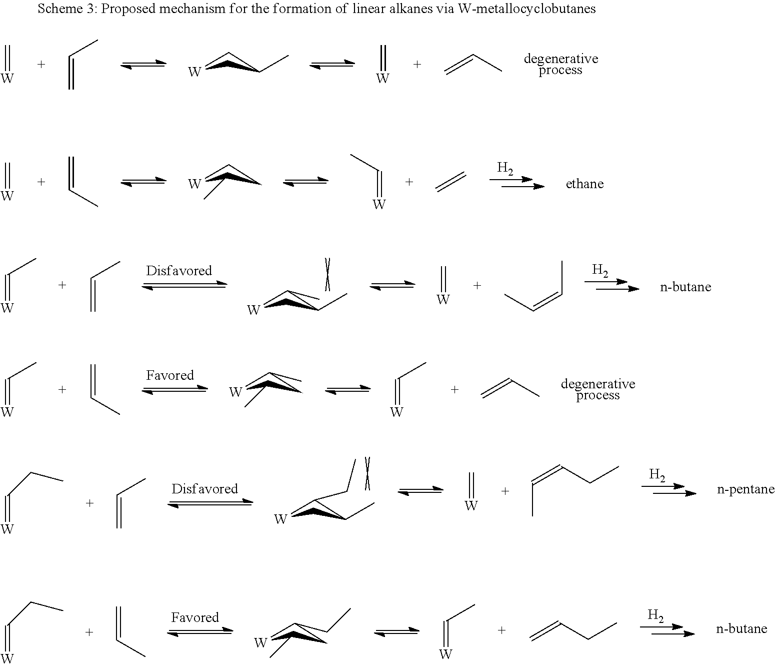

In a batch reactor at 150° C., metathesis of n-decane produces a broad distribution of linear alkanes ranging from methane to triacontane (C30). These linear alkanes were assigned (by GC and GC-MS) according to their retention time and fragmentation pattern by comparison with available references.

### NMR Studies of the Thermal Transformation of 2

The above observations suggest that the reaction proceeds through a W-methylidene intermediate. In order to induce the formation of this species, and in the hope of isolating the methylidene, the thermal stability of 2 in the absence of substrate in situ was studied by solid state NMR.

Heating a supported sample of 2 which was enriched in ¹³C (>95%) from 298 to 345 K, leads to the observation of several new NMR signals. By maintaining the temperature at 345 K for 12 h, most of the ≡Si—O—W(Me)₅ had converted. The spectra of the converted material suggest that the products are the W-methyl/methylidyne species 5 and 6 in scheme 4.

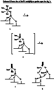

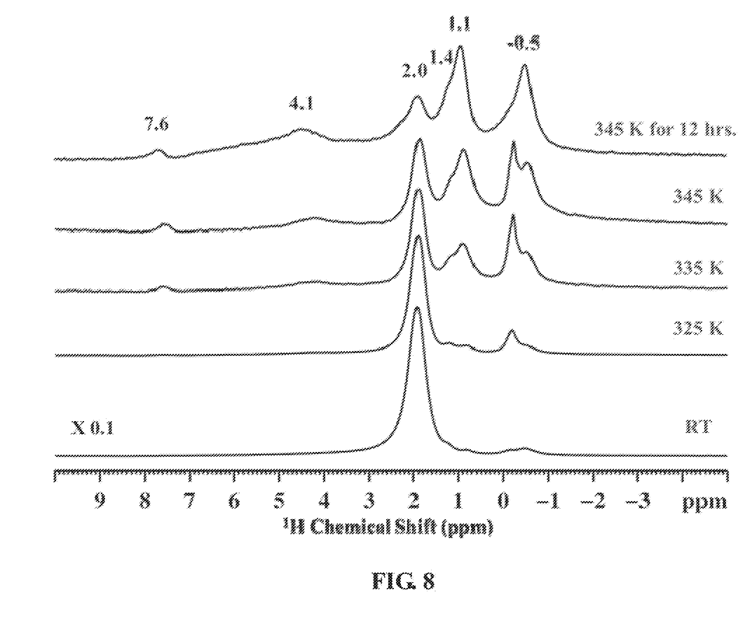

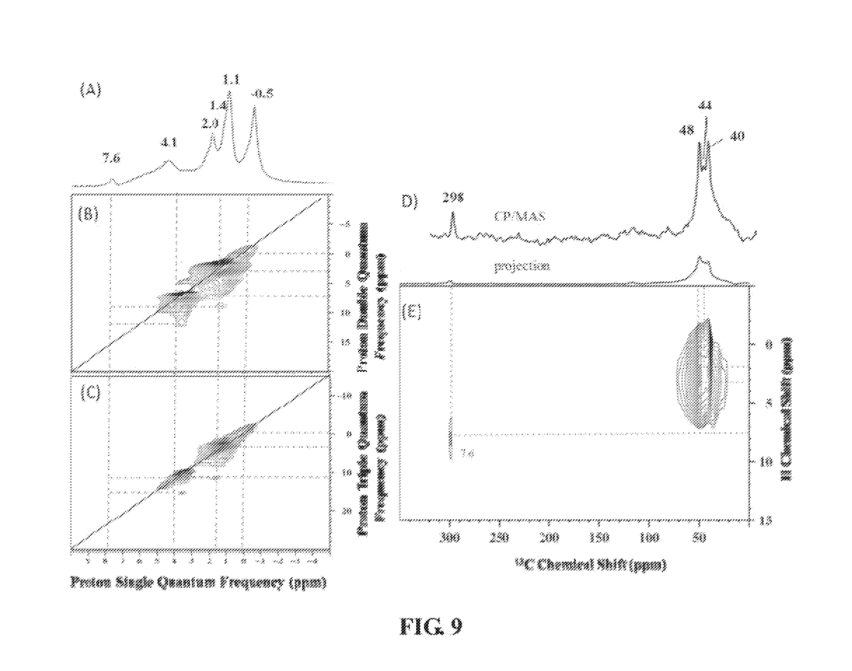

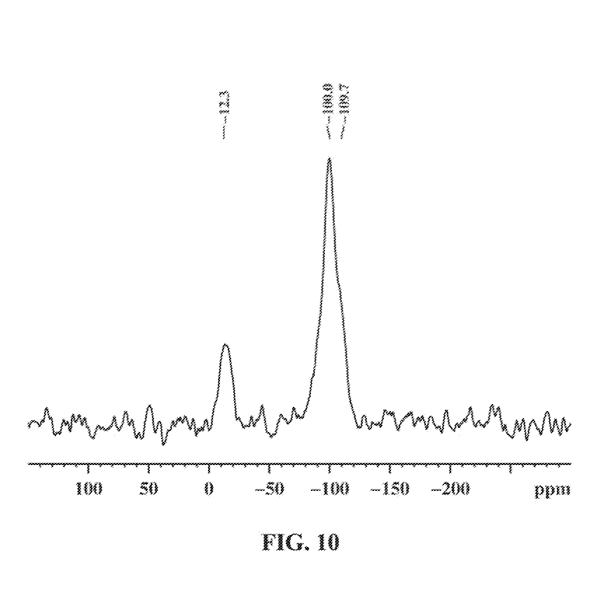

In the converted material the ¹H NMR spectrum (FIG. 8) exhibits four major new signals at 1.1, 1.4, 4.1 and 7.6 ppm. The signals at 1.1, 1.4 and 4.1 ppm auto-correlate in 2D DQ and TQ ¹H-¹H homonuclear dipolar correlation spectra, and are assigned to different methyl groups (FIG. 9B and FIG. 9C). The proton resonance at 7.6 ppm displays no auto-correlation in the DQ and TQ spectra (FIG. 9B). The broad signal at −0.5 ppm is assigned to methane and methyl groups transferred to silica (i.e., ≡SiMe), which is supported by an autocorrelation in DQ and TQ (FIG. 9B and FIG. 9C) and also by ²⁹Si CP/MAS NMR (peak at −12 ppm) (FIG. 10). The signal at 2.0 ppm likely corresponds to unreacted silanol. The ¹³C CP/MAS NMR spectrum (FIG. 9D) displays three signals at 40, 44, and 48 ppm and at lower frequency a signal at 298 ppm is observed. Additionally, the 2D ¹H-¹³C HETCOR NMR spectrum (FIG. 9E) with a short contact time (0.2 ms) shows a correlation between the methyl protons (1.4 and 1.1 ppm) and these two carbon atoms (44 and 40 ppm) respectively, and a correlation between the methyl protons centred at 4.1 ppm with the carbon at 48 ppm allows the assignment of the carbon-proton pairs to the individual methyl groups. Furthermore, the strong correlation between the carbon and proton signals at 298 ppm and 7.6 ppm, respectively, strongly supports the assignment of a methylidyne moiety (W≡CH) (FIG. 9E). Chemical shift values for ¹H and ¹³C in this range are also reasonably consistent with DFT calculations for a model silica surface-supported system that contains a W≡CH functional group.

FIG. 10 shows ²⁹Si CP-MAS NMR spectrum of a mixture of monopodal and bipodal (≡SiO)ₓW≡CH(CH₃)_{y} acquired at 400 MHz with a 5 kHz MAS frequency of 5 kHz. The number of scans was 20 000, and the recycle delay was set to 5 s. A cross polarization time of 5 ms was used. An exponential line broadening of 100 Hz was applied prior to Fourier transform

Furthermore, a correlation in DQ/SQ NMR correlation spectrum between the ≡SiCH₃ at −0.5 ppm and the methyl groups at 4.1 ppm supports transfer of a methyl group to the silica and suggests the formation of bipodal species 6 (¹³C: 48 ppm; ¹H: 4.1 ppm) (Scheme 4). Since no correlation with the other two methyl groups is observed, these two inequivalent methyl groups (¹³C: 44 and 40 ppm; ¹H: 1.4 and 1.1 ppm) can be assigned to the monopodal species 5. The methyl groups of both species 5 and 6 correlate with the methylidyne moiety as observed in both DQ and TQ NMR experiments (FIG. 9B and FIG. 9C).

### Formation of W-Methyl/Methylidyne Species Supports Transient Methylidene Intermediates.

Together, these studies show that 2 evolves upon thermal treatment into a mixture of unprecedented mono and bipodal W-methyl/methylidyne species. This plausibly supports the formation of a transient W-methylidene intermediate 4 (Scheme 4). The grafted WMe₆ species can evolve into a W-methylidyne containing species, which would not be otherwise observable in a comparable homogenous system. See, for example, Chiu, K. W. et al., A. J. Chem. Soc., Dalton Trans. 1981, 1204-1211, which is incorporated by reference in its entirety. These supported W-methylidyne species 5 and 6 were also used as precursors for propane metathesis and produced ethane and butane with traces of methane and pentanes with a TON of 50 after 120 hours at 150° C. They are less active than the pentamethyl compound 2. This can be due to the presence of less methyl groups. If the first step in the process was a bond activation, it would then be easier for species 2 than species 5 or 6.

### Macrocyclic Alkane Synthesis

Transition metal alkylidene species are involved in olefin metathesis and assumed to be key intermediates in alkane metathesis. See, J. M. Basset, C. Coperet. D. Soulivong, M. Taoufik and J. T. Cazat, Acc. Chem. Res., 2010, 43, 323-334, and F. Rascon and C. Coperet, J. Organomet. Chem., 2011, 696, 4121-4131, each of which is incorporated by reference in its entirety. Alkane metathesis is a reaction widely studied employing two catalytic systems: dual catalysts operating in tandem (see, M. C. Haibach, S. Kundu, M. Brookhart and A. S. Goldman, Acc. Chem. Res., 2012, 45, 947-958, which is incorporated by reference in its entirety) and single supported multifunctional catalysts. For the single catalytic system, it is generally assumed that a metal alkylidene hydride or a metal alkylidene alkyl belonging typically to groups V and VI is needed to convert alkanes. This transformation occurs via a multistep mechanism (C—H bond activation, olefin metathesis). Olefins were found to be key intermediates in this reaction forming metallacyclobutanes. See, J. M. Basset. C. Coperet, L. Lefort, B. M. Maunders, O. Maury, E. Le Roux, G. Saggio, S. Soignier, D. Soulivong, G. J. Sunley, M. Taoufik and J. Thivolle-Cazat, J. Am. Chem. Soc., 2005, 127, 8604-8605, and M. Leconte and J. M. Basset, J. Am. Chem. Soc., 1979, 101, 7296-7302, each of which is incorporated by reference in its entirety.) In the past, several approaches to synthesize W methylidene species have been used. Initially, it was postulated that direct protonation of the carbynic W≡C bond by surface Bronsted acids should provide methylidene tungsten species. See, R. Buffon, M. Leconte, A. Choplin and J. M. Basset, J. Chem. Soc., Chem. Commun., 1993, 361-362, and R. Buffon, M. Leconte, A. Choplin and J. M. Basset, J. Chem. Soc., Dalton Trans., 1994, 1723-1729, each of which is incorporated by reference in its entirety. The lack of results for this approach leads to the direct substitution of one ligand of a complex already possessing the alkylidene moiety, followed by cycloaddition of ethylene. See, F. Blanc. R. Berthoud, C. Coperet, A. Lesage, L. Emsley, R. Singh, T. Kreickmann and R. R. Schrock, Proc. Natl. Acad. Sci. U.S.A., 2008, 105, 12123-12127, which is incorporated by reference in its entirety. Non-alkyl ligands (imido, oxo, and phenolate) are generally required to stabilize these alkylidene species explaining that these surface organometallic species are generally restricted for olefin metathesis. See, M. P. Conley, V. Mougel, D. V. Peryshkov, W. P. Forrest, D. Gajan, A. Lesage, L. Emsley, C. Cope'ret and R. R. Schrock, J. Am. Chem. Soc., 2013, 135, 19068-19070, which is incorporated by reference in its entirety. Additionally, direct methanation of the W polyhydrides complex followed by α-H abstraction from the methyl ligand provides encouraging results for obtaining the W methylidene complex. K. C. Szeto, S. Norsic, L. Hardou, E. Le Roux, S. Chakka, J. Thivolle-Cazat, A. Baudouin, C. Papaioannou, J. M. Basset and M. Taoufik, Chem. Commun., 2010, 46, 3985-3987, which is incorporated by reference in its entirety. Therefore, as shown in scheme 2 in Examples, a well-defined silica supported W methyl catalysis can be used. Upon thermal treatment, W pentamethyl complex of species 2 of Scheme 5 possessing no β-H can be transformed into W methylidyne of species 5 and 5′ of Scheme 5. See also, M. K. Samantaray, E. Callens, E. Abou-Hamad, A. J. Rossini, C. M. Widdifield, R. Dey, L. Emsley and J.-M. Basset, J. Am. Chem. Soc., 2014, 136, 1054-1061, which is incorporated by reference in its entirety.

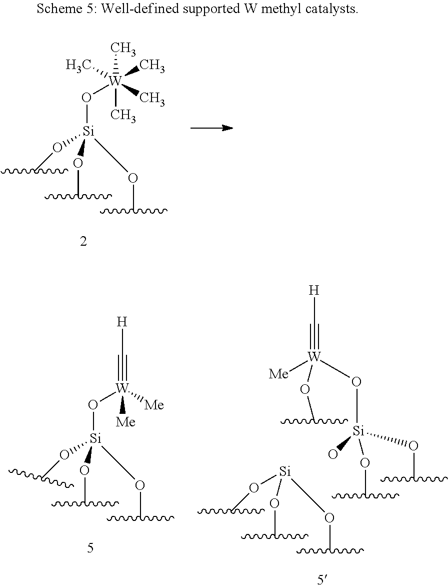

These species were found to be active for propane metathesis giving lower and higher linear homologues. These results are in contrast to those obtained for the silica supported Schrock complex possessing the neopentyl/neopentylidyne group which is found to be much less active for propane metathesis than species 5. See, E. Le Roux, M. Taoufik, A. Baudouin, C. Coperet, J. Thivolle-Cazat, J. M. Basset, B. M. Maunders and G. J. Sunley, Adv. Synth. Catal., 2007, 349, 231-237, which is incorporated by reference in its entirety. Nevertheless, this complex (≡SiO)W(≡CtBu)CH₂tBu)₂ was very active for the propene metathesis. See, E. Le Roux, M. Taoufik, M. Chabanas, D. Alcor, A. Baudouin, C. Coperet, J. Thivolle-Cazat, J. M. Basset, A. Lesage, S. Hediger and L. Emsley, Organometallics, 2005, 24, 4274-4279, which is incorporated by reference in its entirety. To account for the observed reactivity, the formation of a W bis-alkylidene was suggested without experimental evidence.

Disclosed herein is the isolation and characterization at the molecular level of the well-defined W bis-methylidene methyl species promoted by PMe₃ from tautomerization of W

methylidyne methyl species 5. Its activity towards cycloalkane metathesis is also disclosed.

Xue and co-workers have observed by NMR spectroscopy that W-alkyl % alkylidyne with a pendent silyl group could undergo a tautomerization via an α-Hmigration to form a d⁰Wbis(alkylidene) species in a homogeneous phase. See, L. A. Morton. R. T. Wang, X. H. Yu, C. F. Campana, I. A. Guzei, G. P. A. Yap and Z. L. Xue, Organometallics, 2006, 25, 427-4, L. A. Morton, S. J. Chen, H. Qiu and Z. L. Xue, J. Am. Chem. Soc., 2007, 129, 7277-7283. Z. L. Xue and L. A. Morton, J. Organomet. Chem., 2011, 696, 3924-3934, and K. G. Caulton, M. H. Chisholm, W. E. Streib and Z. L. Xue, J. Am. Chem. Soc., 1991, 113, 6082-6090, each of which is incorporated by reference in its entirety. Additionally, they calculated by DFT that the equilibrium between the hypothetical molecular Me₃W≡CH complex and its corresponding W his-methylidene has an energy barrier of only 5 kcal/mol at room temperature (see Scheme 6). See, L. A. Morton, X. H. Zhang, R. T. Wang, Z. Y. Lin, Y. D. Wu and Z. L. Xue, J. Am. Chem. Soc., 2004, 126, 10208-10209, which is incorporated by reference in its entirety.

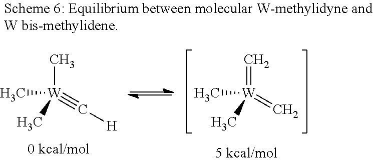

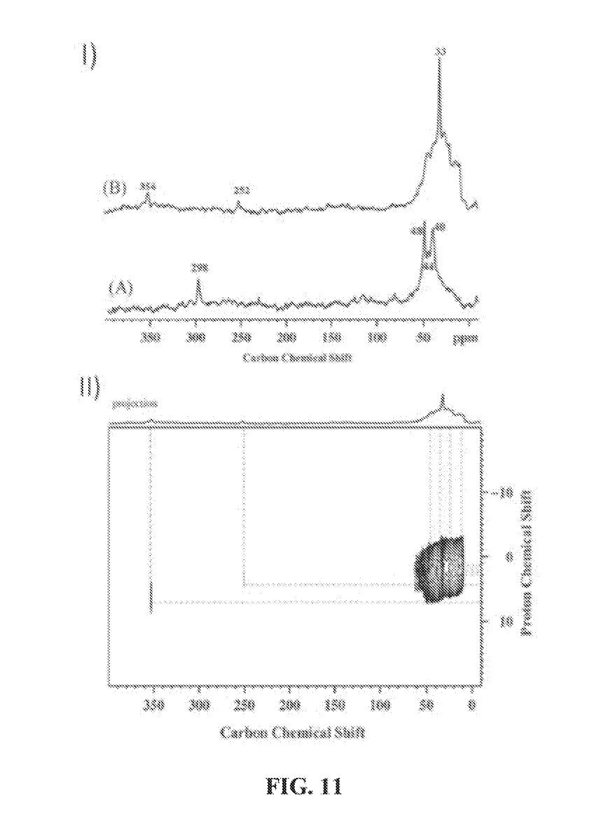

Furthermore, they found that the equilibrium between these W alkylidyne alkyl and W bis-alkylidene species could be catalysed by coordination of trimethylphosphine. Addition of PMe₃ on (Me₃SiCH₂)₃W≡CSiMe₃ promotes an observable exchange to give W bis-alkylidene tautomers (Me₃SiCH₂)₂W(═CHSiMe₃)₂(PMe₃) and (Me₃SiCH₂)₃W≡CSiMe₃(PMe₃) at room temperature. See, L. A. Morton, X. H. Zhang, R. T. Wang, Z. Y. Lin, Y. D. Wu and Z. L. Xue, J. Am. Chem. Soc., 2004, 126, 10208-10209, which is incorporated by reference in its entirety. Thus, species 5 and 5′ could evolve into W bis-methylidene species via H-transfer of a pendent methyl ligand under the disclosed alkane metathesis conditions. Species 5 and 5′ were fully characterized by NMR spectroscopy in FIG. 11: the ¹³C CP/MAS spectrum displays four signals at 298, 48, 44 and 40 ppm (FIG. 11-IA). The carbon signal at 298 ppm, confirmed by DFT calculations, is assigned to a methylidyne moiety (W≡CH), in which the corresponding proton signal shows no autocorrelation in 1H-1H double quantum and triple quantum. The carbon resonances at 44 and 48 ppm correspond to the two methyl groups of monopodal species 5 and the carbon signal at 48 ppm corresponds to the methyl group of bipodal species 5′.

In the work described herein see E. Callens, E. Abou-Hamad, N. Riache and J. M. Basset, Chem. Comm., 2014, 50, 3982-3985 a vapor pressure of PMe₃ was introduced on silica supported ¹³C enriched 5 and 5′. ¹³C CP/MAS solid state NMR spectroscopy of the resulting powder shows the disappearance of the signal at 298 ppm and the appearance of two signals at 356 ppm and 252 ppm (FIG. 11-IB). The other signal at 33 ppm corresponds likely to PMe₃ physisorbed on dehydroxylated silica. Furthermore, the carbon resonance at 252 ppm shows a correlation with proton chemical shifts centered at 4.2 ppm in the 2D ¹H-¹³C heteronuclear (HETCOR) NMR experiment (FIG. 11-II) with a short contact time (0.2 ms), attributed to a typical W-alkylidene species. The carbon resonance at 356 ppm correlates with the proton chemical shifts centered at 7 ppm (FIG. 11-II), which corresponds to the W methylidyne species. These observed ¹³C chemical shifts match with those obtained for W methylidyne and methylidene species in the liquid phase of the molecular complex (Me₃SiCH₂)₃W≡CSiMe₃; in the presence of PMe₃ in toluene d₈. See, L. A. Morton, X. H. Zhang, R. T. Wang, Z. Y. Lin. Y. D. Wu and Z. L. Xue, J. Am. Chem. Soc., 2004, 126, 10208-10209, which is incorporated by reference in its entirety. Note that several resonances centered at 33 ppm correlating with protons between 1.9 and 1.2 ppm correspond probably to different orientations of the methyl groups.

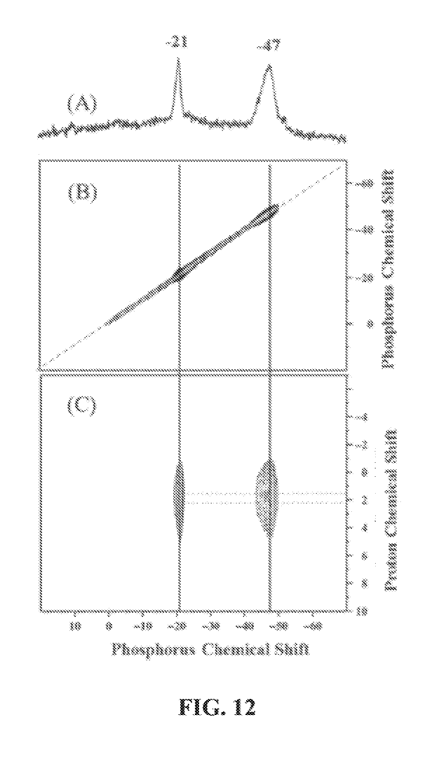

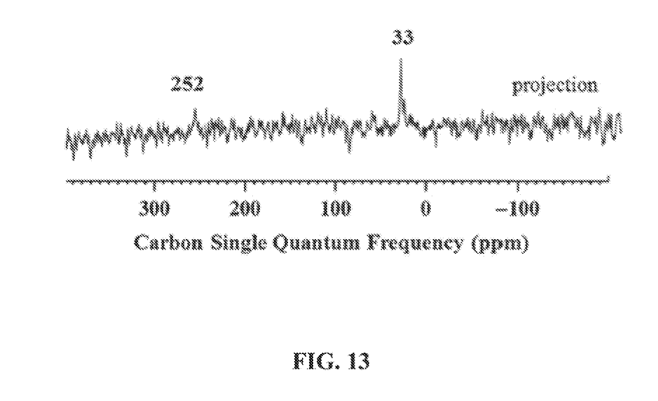

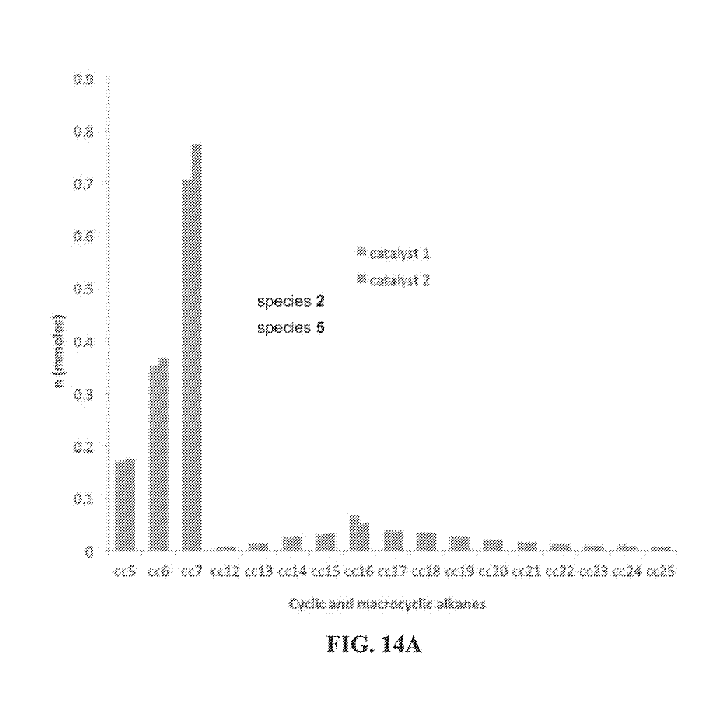

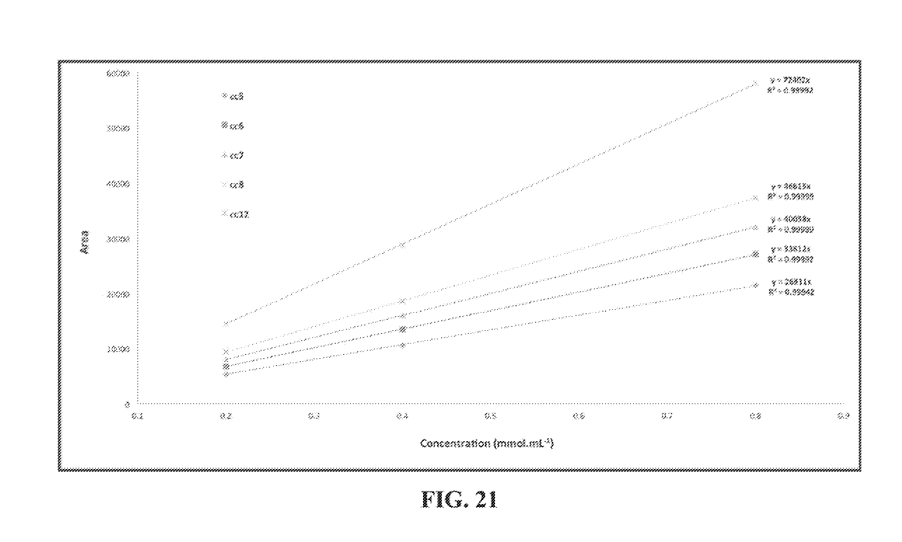

Additionally, ³¹P solid state NMR spectroscopy was also undertaken since its natural isotopic abundance allows fast acquisition. The ³¹P NMR spectrum shows two signals at −21 and −47 ppm (FIG. 12A). The latter corresponds to the PMe₃ physisorbed on silica. Thus, the phosphorous resonance at −21 ppm corresponds likely to an average of the different W supported tautomer species coordinated with PMe₃. The ³¹P-³¹P spin-diffusion shows no correlation between the two different phosphorus signals, confirming the existence of two distinct coordination sites of PMe₃: coordination to the W atom and physisorption on silica (FIG. 21B). See, K. Takeda, K. Takegoshi and T. Terao, J. Chem. Phys., 2002, 117, 4940-4946, and K. Takegoshi, S. Nakamura and T. Terao, Chem. Phys. Lett., 2001, 344, 631-637, each of which is incorporated by reference in its entirety. While, in the 2D ¹H-³¹P HETCOR NMR spectrum (FIG. 12C) with a contact time of 1 ms the two ³¹P signals at −21 and −47 ppm correlate with methyl protons between 1.6 and 2.1 ppm. To determine whether two phosphorous ligands could coordinate to the W metal center, we also performed ID INADEQUATE (Incredible Natural Abundance Double Quantum Transfer Experiment). See. E. Ciampi, M. I. C. Furby, L. Brennan, J. W. Emsley, A. Lesage and L. Emsley, Liq. Cryst., 1999, 26, 109-125, F. Fayon, G. Le Saout, L. Emsley and D. Massiot, Chem. Commun., 2002, 1702-1703, and S. Cadars. J. Sein, L. Duma, A. Lesage, T. N. Pham. J. H. Baltisberger, S. P. Brown and L. Emsley, J. Magn. Reson., 2007, 188, 24-34, each of which is incorporated by reference in its entirety. This is a NMR method to identify pairs of bonded nuclei, including when the two nuclei have the same isotropic chemical shift. See. L. Duma, W. C. Lai, M. Carravetta, L. Emsley, S. P. Brown and M. H. Levitt, ChemPhysChem, 2004, 5, 815-833, 25 F. Fayon, D. Massiot. M. H. Levitt, J. J. Titman, D. H. Gregory, L. Duma, L. Emsley and S. P. Brown, J. Chem. Phys., 2005, 122, 194313, M. M. Maricq and J. S. Waugh, J. Chem. Phys., 1979, 70, 3300-3316, and D. Gajan. D. Levine. E. Zocher, C. Coperet, A. Lesage and L. Emsley, Chem. Sci., 2011, 2, 928-931, each of which is incorporated by reference in its entirety. The ID refocused INADEQUATE spectrum shows no signal at −21 ppm. This result strongly supports that only one molecule of PMe₃ is coordinated per tungsten. Schrock and Clark reported that (Me₃CCH₂)₃W≡CCMe₃ reacts with neat PMe₃ to form (Me₃CCH₂)—W(═CHCMe₃)(≡CCMe₃)(PMe₃)₂ through CMe₄ elimination at 100° C. in a sealed tube. See, D. N. Clark and R. R. Schrock, J. Am. Chem. Soc., 1978, 100, 6774-6776, which is incorporated by reference in its entirety. Thus, the 2D ¹³C—¹³C double-quantum experiment was needed to confirm the assignment of the W bismethylidene supported species. The DQ frequency in the w₁ dimension corresponds to the sum of two single quantum (SQ) frequencies of the two coupled carbon and correlates in the w₂ dimension with the two corresponding carbon resonances. See, M. Feike, D. E. Demco, R. Graf, J. Gottwald, S. Hafner and H. W. Spiess, J. Magn. Reson., Ser. A, 1996, 122, 214-221, which is incorporated by reference in its entirety. Conversely, carbon groups with spins that are not dipolar coupled will give no signals. The projection on the w2 dimension of the ¹³C—¹³C double-quantum MAS spectrum of species 5 in the presence of PMe₃ shows the appearance of a signal at 252 ppm confirming the presence of two neighboring equivalent methylidene groups (FIG. 13).

These results strongly support that grafted W methylidyne species 5 undergoes tautomerization to form W bis-methylidene species 8 in the presence of PMe₃, as shown in Scheme 7. Moreover, adding cyclohexene to species 3a and 3b lead also to the formation of these bis-carbene species.

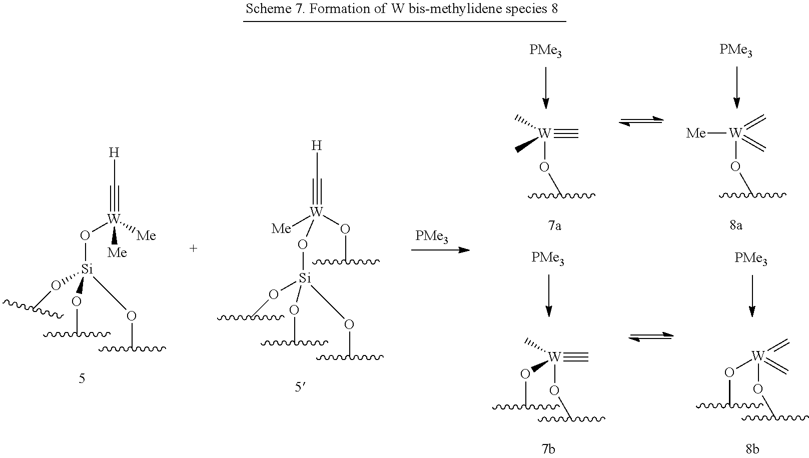

### Distribution of Macrocyclic Alkanes

To have a better understanding of their reactivity, these supported catalysts were studied in the metathesis of cyclooctane. Cyclooctane metathesis can offer a rapid and facile access to the cyclic structures. In 2008, cyclooctane metathesis in a tandem system employing the pincer-ligated iridium complexes acting as hydrogenation/dehydrogenation catalysts combined with Schrock-type Mo alkylidene complexes as olefin metathesis catalyst has been reported. See, R. Ahuja, S. Kundu, A. S. Goldman. M. Brookhart, B. C. Vicente, S. L. Scott, Chem Commun 2008, 253, which is incorporated by reference in its entirety. Although the cyclooctane conversion was 27-80%, this tandem catalytic system suffers from the formation of polymeric products (>80%), which renders difficult the isolation of macrocyclic compounds. Besides, these alkanes correspond essentially to cyclooctane oligomers (cC16, cC24, cC32 and cC40).

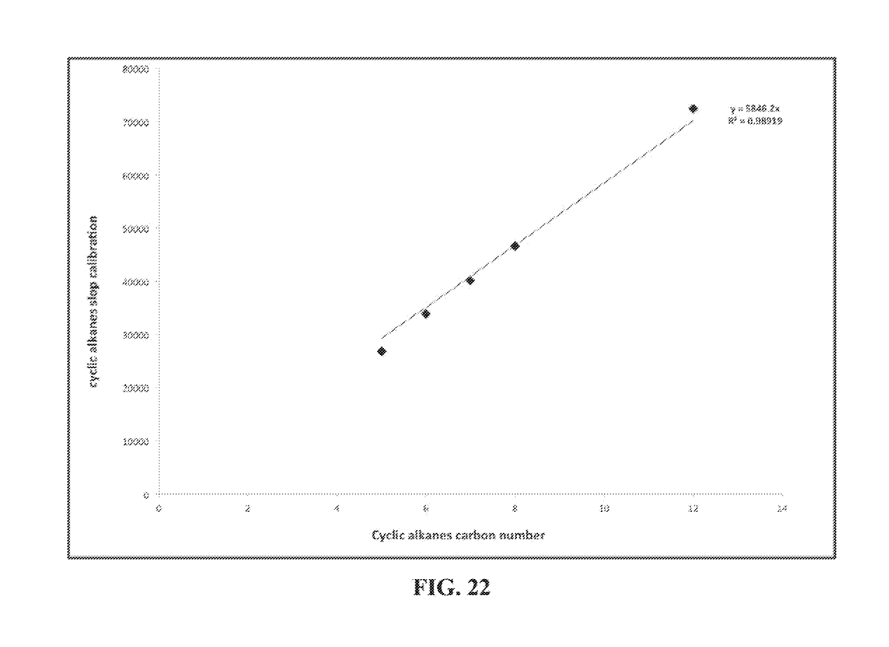

Employing a single multifunctional silica-supported catalyst (e.g. species 2 or 5) can be an alternative catalytic system for synthesis of wider distribution of macrocyclic alkanes. For example, cyclic alkane (3.7 mmol) and catalyst precursor 1 (6.5 μmol) were added via a glove box into an ampoule. Each ampoule was then sealed under vacuum and heated at 150° C. At the end of the catalytic run, the reaction was allowed to cool to −78° C. After filtration, an aliquot was analyzed by GC and GC-MS techniques (for calibration table see FIGS. 21-22). To ensure that the nature of the catalytic site is heterogeneous, the filtrate was analyzed at the end of the reaction and found W concentration less than 0.1 ppm. See, R. H. Crabtree, Chem Rev 2012, 112, 1536, which is incorporated by reference in its entirety. Besides, no reaction could be observed when adding cyclooctane to this filtrate. To analyze the higher oligomers, a suitable GC methodology was developed allowing the detection up to pentamers of cyclooctane (Column HP-5; 30 m length×0.32 mm ID×0.25 μm film thickness; temperature program: 40° C. (1 min), 40-250° C. (15° C./min), 250° C. (1 min), 250-300° C. (10° C./min), 300° C. (15 min), tR (cyclooctane): 6.5 min, tR (cyclohexadecane, dimer): 6.5 (13.6) min, tR (cyclotetradecane, trimer): 19.3 min).

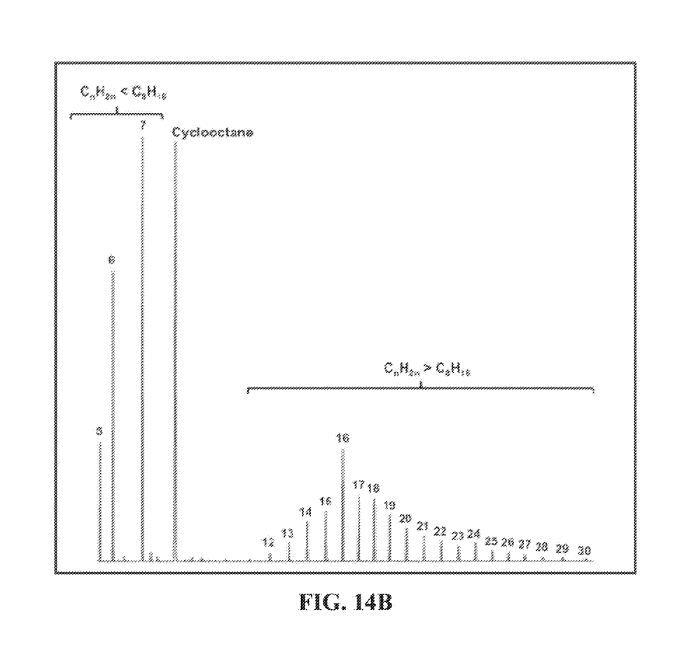

The cyclooctane metathesis reaction using catalyst precursors 2 or 5 is found to be very similar in terms of reactivity and selectivity. TON values are 311 and 362, respectively, for this alkane metathesis after 340 h. Conversions reached 50% and 57%, respectively (FIG. 14A). Supported species 7 and 8 were found to be inactive for this cyclooctane metathesis because an open coordination site is taken by the added phosphine ligand or the strong s-donor property of PMe₃ could decrease the electrophilic character of the W metal.

Typical GC chromatogram of cyclooctane metathesis displays a distribution of peaks. The most intense ones have molecular formula CₙH₂ₙ: i) three peaks with lower retention time than cyclooctane (on GC) correlate with the peaks with lower molecular weight (<C₈) (on GC-MS) and ii) other peaks with longer retention time and higher molecular weight (FIG. 14B).

This cyclooctane metathesis transformation involves the formation of an olefin intermediate that would undergo a metathesis step. Having demonstrated earlier that a cyclooctene would undergo a facile ring opening metathesis polymerization, we studied whether coordination of a cyclohexene (well-known to be difficult for ROMP; see, G. Natta, G. Dallasta, I. W. Bassi and G. Carella, Makromol. Chem., 1966, 91, 87-106, which is incorporated by reference in its entirety) on the W metal sphere could also evolve into a W bis-methylidene species. Contact of the cyclohexene with 2 leads to several carbon resonances at 307, 252, 144, 59 and 44 ppm in ¹³C NMR spectroscopy. The signal at 252 ppm indicates the presence of two methylidene ligands, demonstrating that an olefin could act as PMe₃ by promoting the tautomerization. The signals at 307 and 142 ppm are respectively assigned to methylidyne moiety (W≡CH) and the CH of the sp² carbons of cyclohexene. The one at 59 could correspond to a W-metallacycle adopting a square bipyramidal geometry and the methyl groups at 44 pm.

Extensive solid-state NMR analysis provides the evidence of the first supported W bis-methylidene species, upon treatment of supported W methylidyne with either PMe₃ or an olefin. These results are important for a better comprehension of alkane metathesis catalyzed by supported single catalytic system.

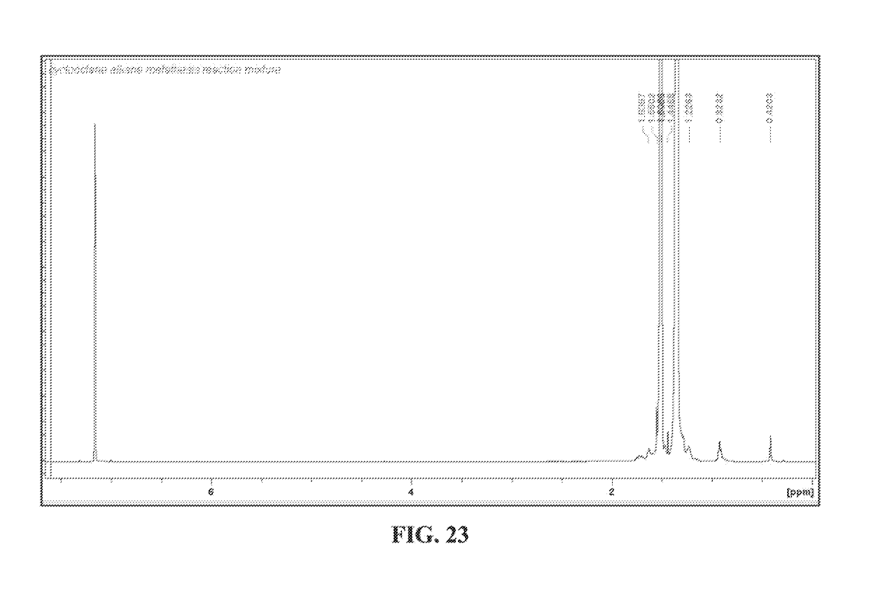

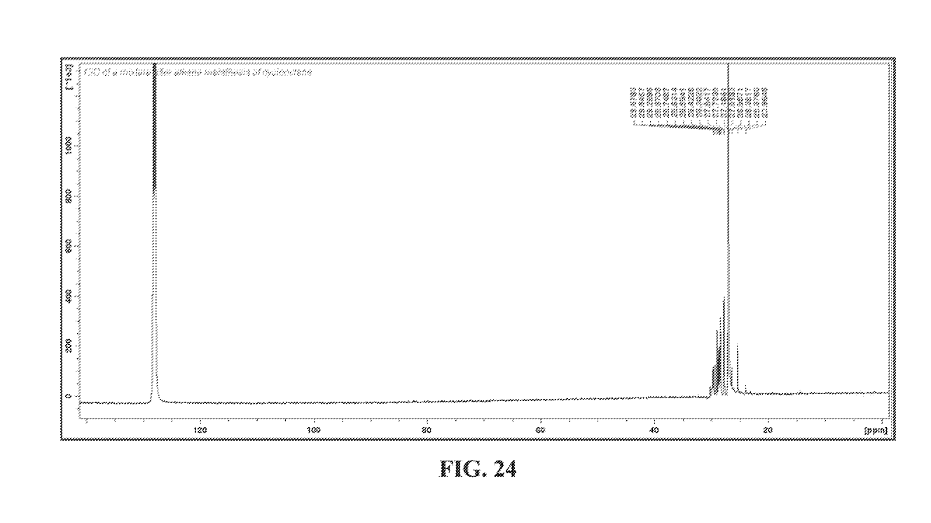

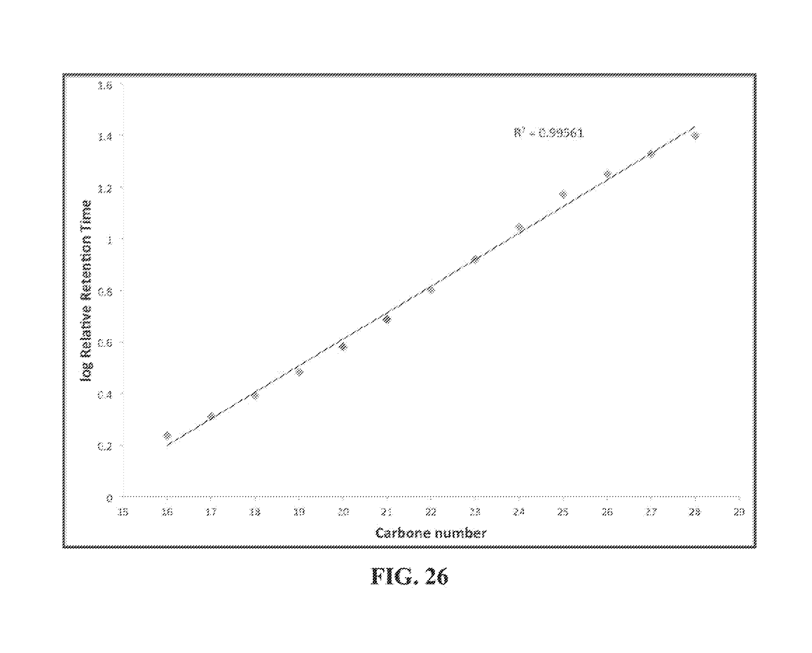

Lower cycloalkanes with molecular weight ranging from C₅ to C₇ are attributed to cyclopentane, cyclohexane and cycloheptane. They result from the ring contraction of cyclooctane (vide infra the mechanism). With very few literature data available, the compounds with chemical formula of CₙH₂ₙ ranging from C₁₂ to C₄₀ required more thoughtful characterizations. From molecular formula, they could be either macrocyclic alkanes or linear olefins as well as branched cyclic alkanes. Firstly, proton and carbon NMR of the resulting solution at the end of the catalytic run shows the absence of olefinic protons and sp² carbons which would correspond to a double bond (FIGS. 23-24). Macrocyclic alkanes from C₁₂-C₁₅, C₂₄, C₂₈ and C₃₀ were identified by comparison with mass spectrum of the corresponding library references (NIST Standard Reference Database, http://webbook.nist.gov/chemistry/). They exhibit similar fragmentation pattern and ion ratio. However, no EI spectra library was found for most of the other alkanes requiring ion fragmentation interpretation. For most of alkane products in the range of C₁₂ to C₄₀ showed similar ion fragmentation pattern. The comparison between their ion fragmentation pattern with the only cycloeicosane (cC₂₀) and cycloheneicosane (cC₂₁) patterns disclosed in literature (see, Y. L. Wang, X. M. Fang. Y. Bai, X. X. Xi, S. L. Yang, Y. X. Wang, Org Geochem 2006, 37, 146, which is incorporated by reference in its entirety) supports that C₂₀ and C₂₁ from the mixture are macrocyclic alkanes and by extent strongly support that the other alkanes from C₁₂ to C₄₀ belong to this same family. Secondly, the correlation of the logarithm of the relative retention time versus the carbon atom numbers, known as Kovats retention index (see, N. H. Ray, J Appl Chem 1954, 4, 21, which is incorporated by reference in its entirety), was examined. The experimental linear correlation found (0.996) corroborates with the assignment for macrocyclic alkanes series as major products (FIG. 26).

C₁₂₋₁₅, C₂₄, C₂₈ and C₃₀ were easily assigned to macrocyclic alkanes using library references (see, NIST Standard Reference Database, webbook.nist.gov/chemistry). However, for most of the other alkanes, no library match EI spectra are disclosed to our knowledge, thus, intensive ion fragmentation interpretation was required. The mass spectra of only two macrocyclic alkanes were disclosed in literature to date: cycloeicosane and cycoheneicosane. See, Wang, Y. L.; Fang, X. M.; Bai, Y.; Xi, X. X.; Yang, S. L.; Wang, Y. X. Org Geochem 2006, 37, 146, and Audino, M.; Grice, K.; Alexander, R.; Kagi, R. I. Org Geochem 2001, 32, 759, each of which is incorporated by reference in its entirety. Comparison of their fragmentation with C₂₀ and C₂₁ from cyclooctane metathesis confirms their assignment to macrocyclic alkanes. Moreover, comparison of ion fragmentation pattern of compounds from C₁₂-C₃₀ with existing macrocyclic alkane ones seems that likely they correspond to macrocyclic alkanes. The plot of the log of the relative retention time versus carbon numbers for the alkanes in the range C₁₇-C₂₉ shows a correlation (0.996) (FIG. 26). As a linear correlation between logarithm of the relative retention time and carbon number is known for a given class of compounds (see, Ray, N. H. J Appl Chem 1954, 4, 21, which is incorporated by reference in its entirety), these results strongly support that this mixture corresponds to a common series of compound attributed to macrocyclic alkanes.

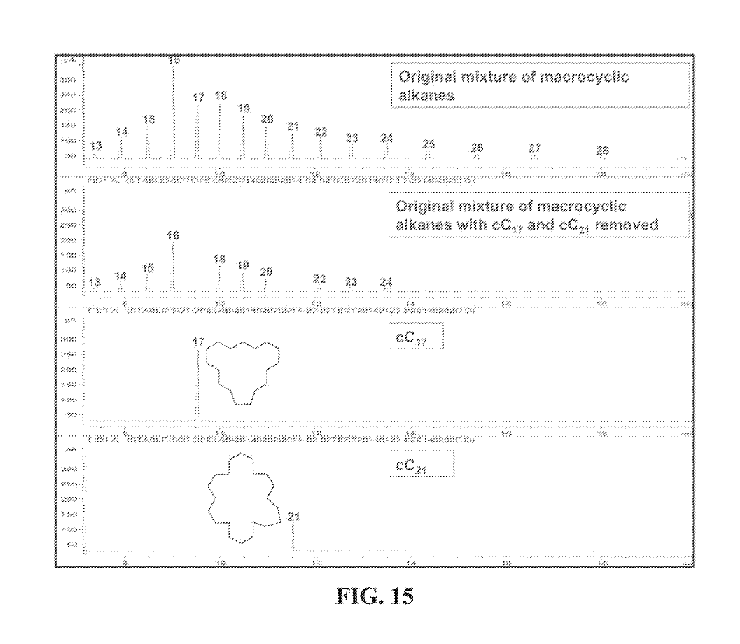

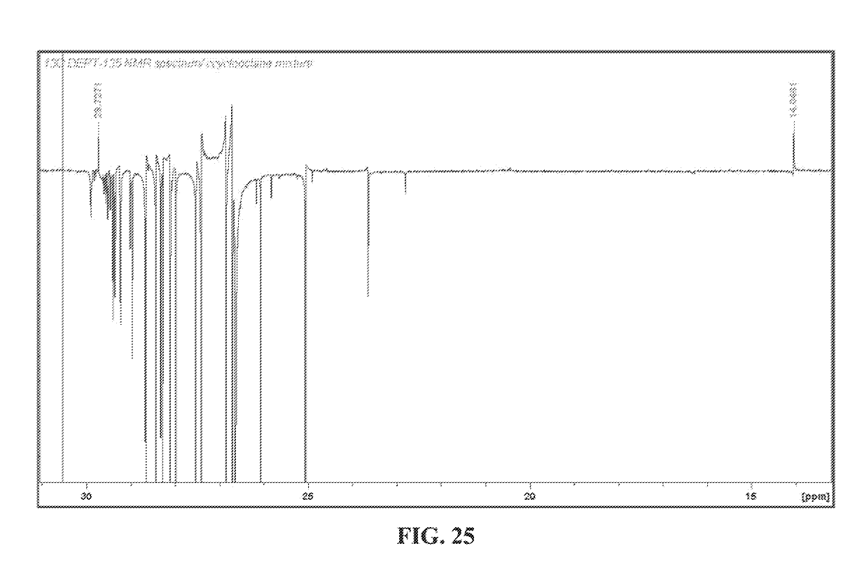

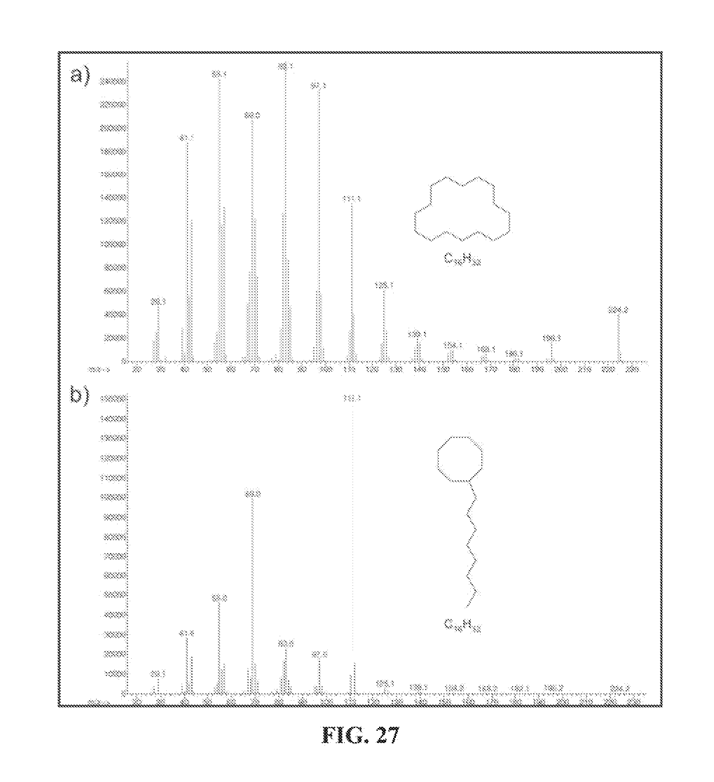

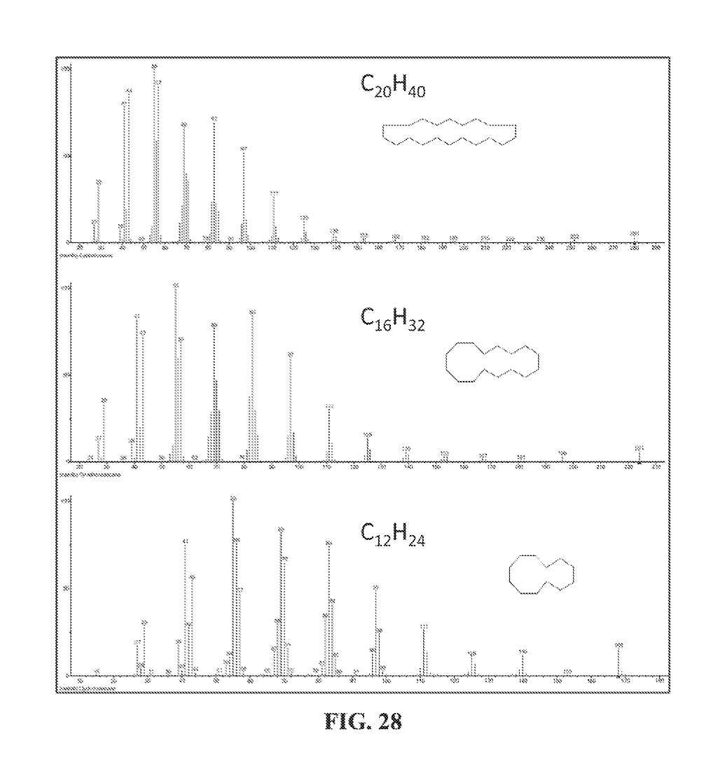

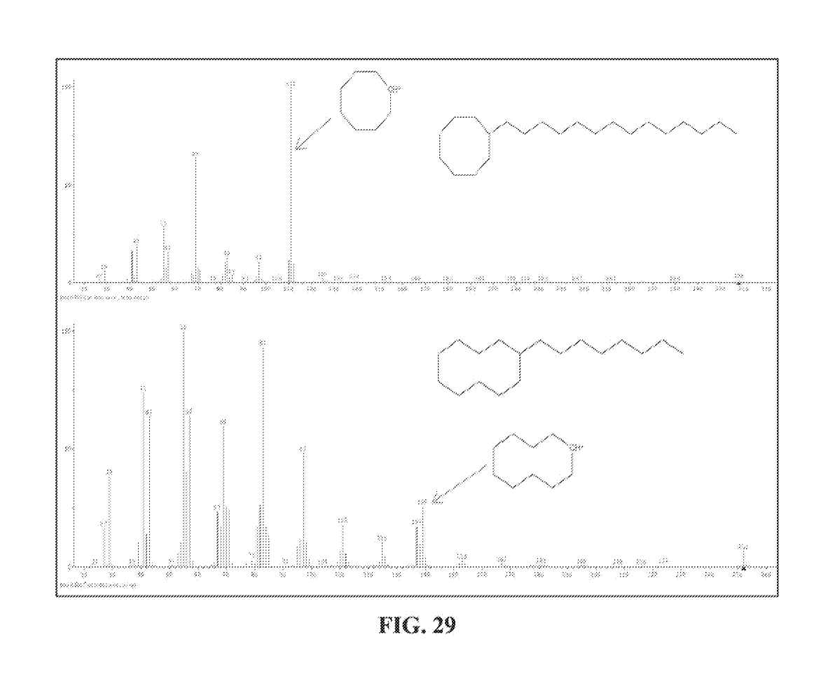

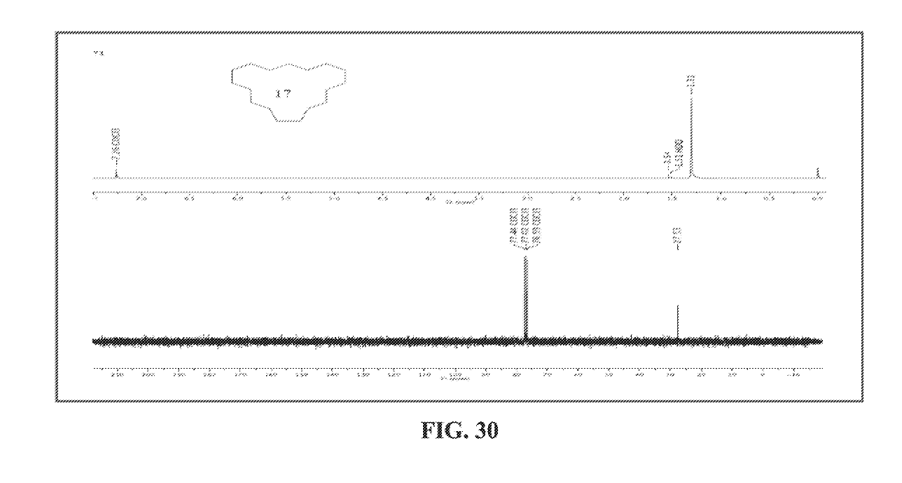

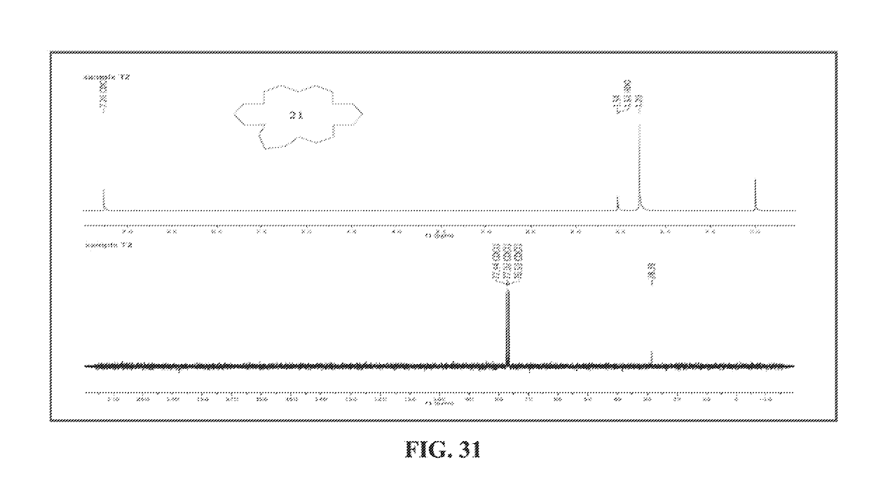

In addition to ¹H and ¹³C NMR spectroscopies, the distortionless enhancement by polarisation transfer (DEPT-135) NMR of the reaction mixture displays weak signals corresponding to CH and CH₃ groups suggesting also the presence of substituted cyclic alkanes or linear alkanes (FIG. 25). To unambiguously distinguish between the pure macrocyclic alkanes and the branched ones, the ion fragmentation of octylcyclooctane and cyclohexadecane was compared. For this purpose, octylcyclooctane was synthesized starting from cyclooctanone. See, W. Giencke, O. Ort, H. Stark. Liebigs Annalen Der Chemie 1989, 671, which is incorporated by reference in its entirety. As expected, octylcyclooctane and cyclohexadecane exhibit different retention times (t_{R}: 13.35 and 13.56 min respectively). More importantly, their ion fragmentation pattern differs significantly (FIG. 27). In fact, the mass spectrum of octylcyclooctane shows low intense molecular ion at m/z 224 and higher intensity of a characteristic ion fragment corresponding to cyclooctane carbocation secondary fragmentation peak at m/z 111, which represents the loss of alkyl chain (see FIGS. 28-29 for EI spectra of cyclic and branched cyclic alkanes). GC preparative fraction collector was employed to isolate two macrocyclic alkanes from the reaction mixture, cycloheptadecane (cC₁₇) and cycloheneicosane (cC₂₁) (FIG. 15). ¹H and ¹³C NMR spectroscopics of these two samples gave respectively single resonance signals (FIGS. 30-31). These experiments confirm unambiguously the structure of cyclooctane metathesis products as purely cyclic compounds.

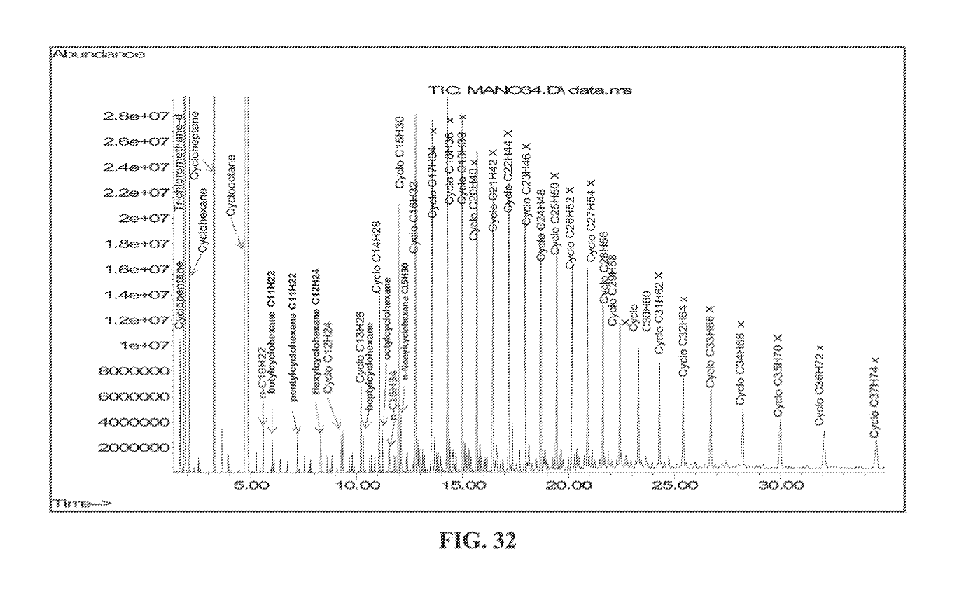

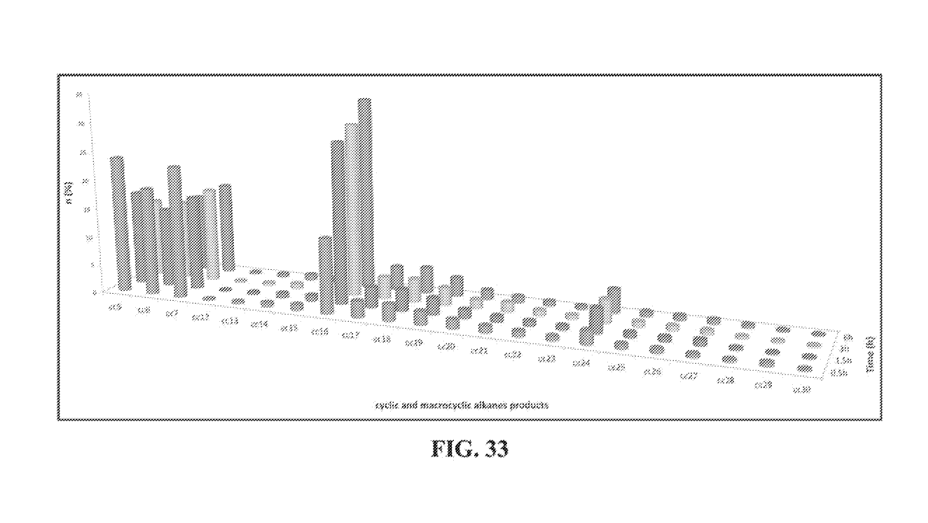

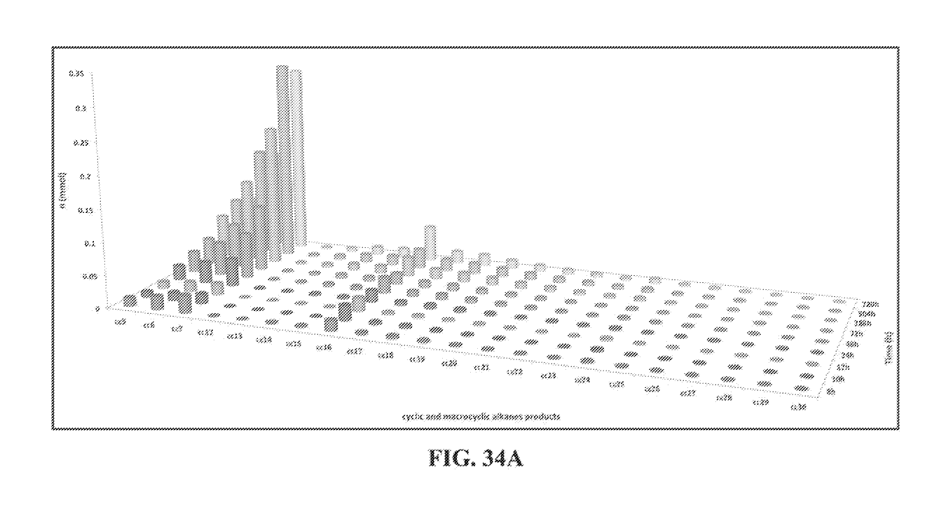

These results demonstrate that the major products of cyclooctane metathesis in the range of C₁₂ to C₄₀ are pure macrocyclic alkanes. A different distribution was observed compared to the tandem catalytic system with a wider distribution of macrocyclic alkanes. Finally, traces of linear alkanes and n-alkyl cyclohexanes compounds were also observed (GC/GC-MS, molar fraction: less than 1% for each family) (FIG. 32).

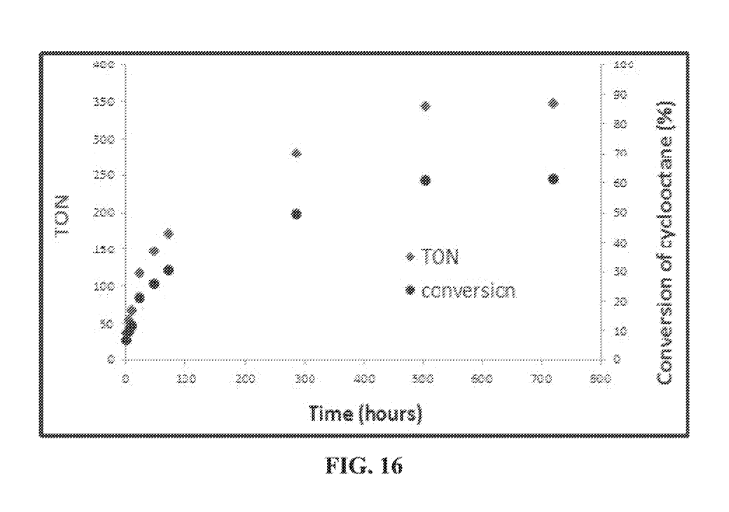

A kinetic study of the cyclooctane metathesis catalyzed by species 2 was carried out at 150° C. The plots of TONs and conversion versus time are given in FIG. 16. A final conversion of 60% is reached with 340 TONs. The catalyst remains active over a long period of time (up to minimum 500 hours) which could correspond to a thermodynamic equilibrium or the deactivation of the catalysts. An initial turnover frequency of 40 mol of cyclooctane (mol_{W})⁻¹h⁻¹ is obtained. Two independent runs confirmed the reproducibility of this catalytic reaction.

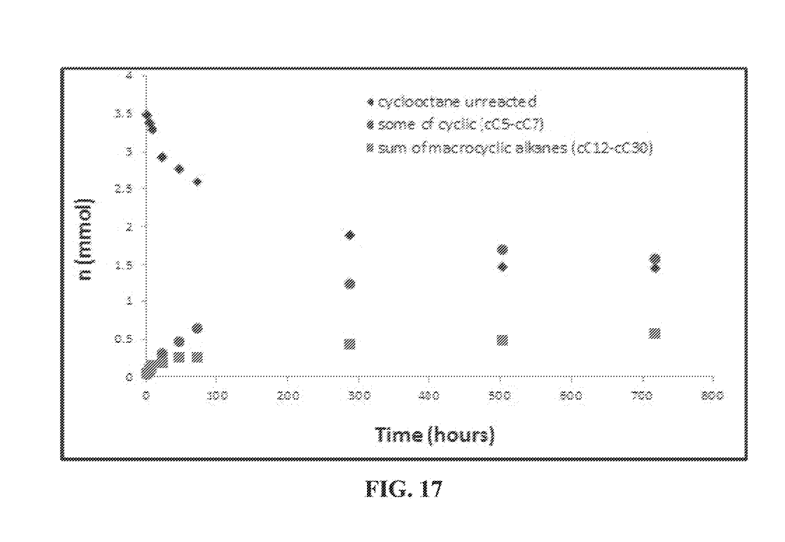

Cyclooctane conversion and cyclooctane metathesis product selectivity (cyclic and macrocyclic alkanes) versus time are showed in FIG. 17. The cyclic/macrocyclic alkane ratio is not constant with time. After 24 h, the plateau corresponding to macrocyclic alkanes is attained. At this time, cyclooctane is likely to be transformed mainly into cyclic alkanes. Above 500 h, 24% of the total number of mol produced corresponds to higher macrocyclic alkanes.

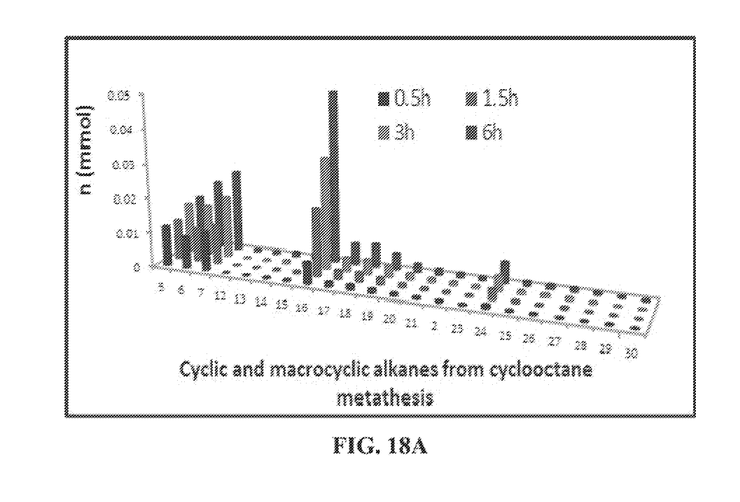

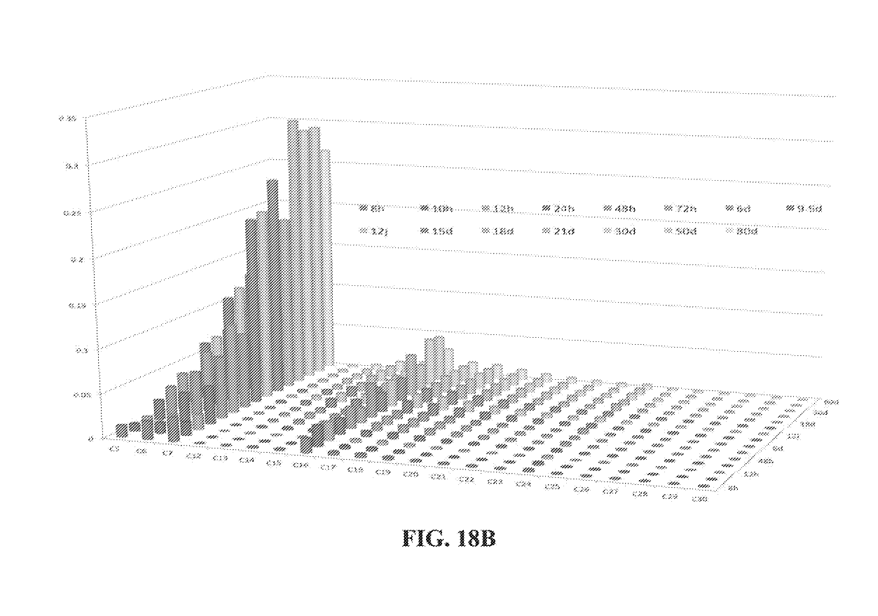

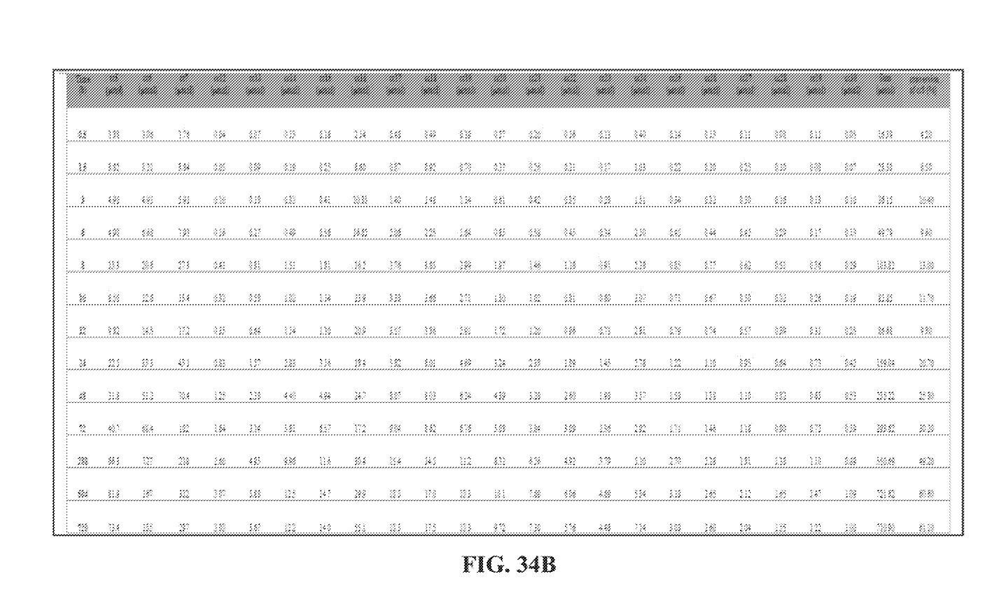

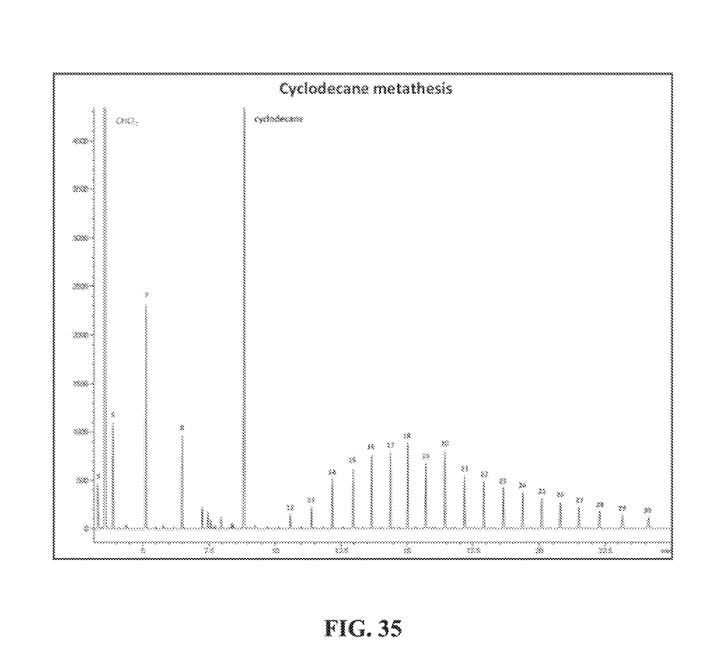

Besides, the first hours of this cyclooctane metathesis were also examined (FIGS. 18 and 34). Interestingly, we found that our W catalytic supported system is selective for the formation for cC₁₆ (cyclic dimer) (molar fraction: 30% for the dimer and up to 60% for all the macrocyclic alkanes). The selectivity toward macrocyclic oligomers decreases with time, which is illustrated by ring contraction at the expense of ring expansion (FIGS. 35A-35B).

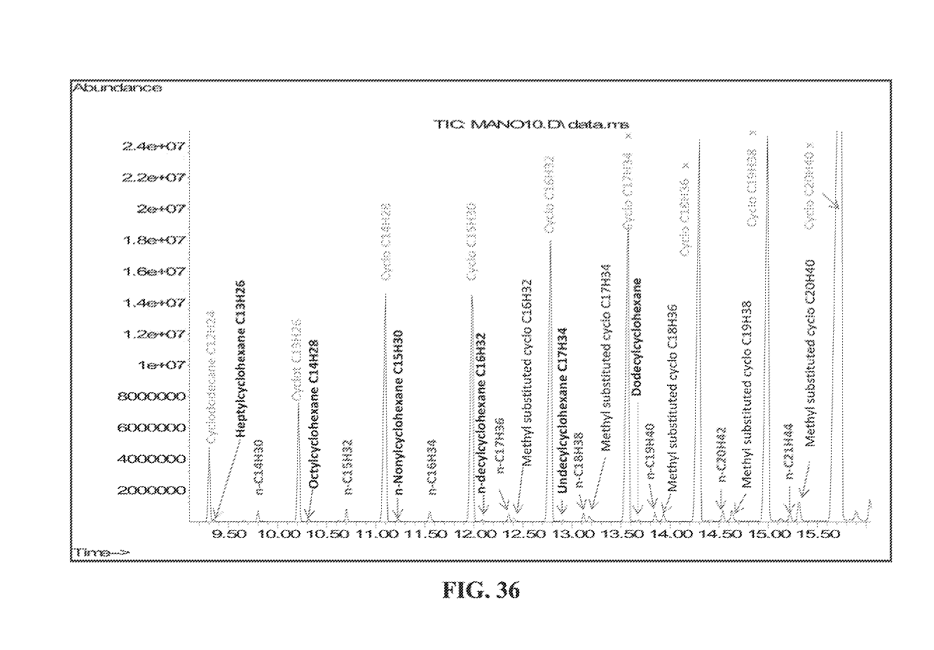

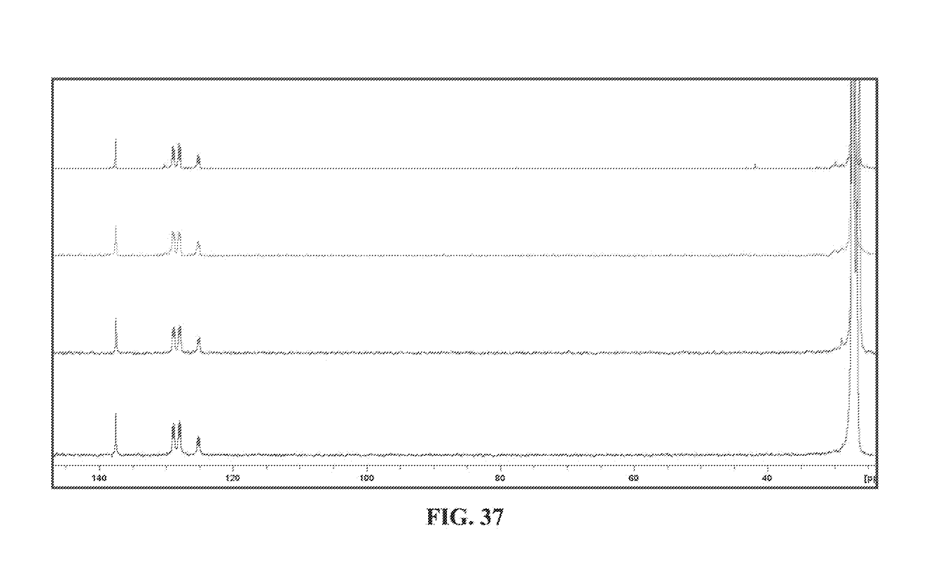

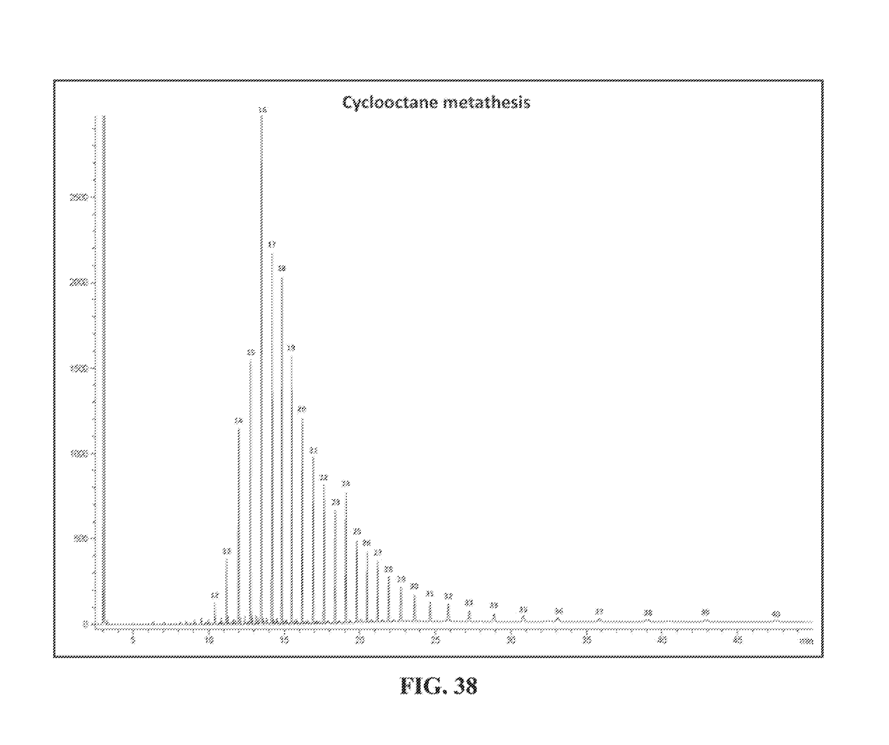

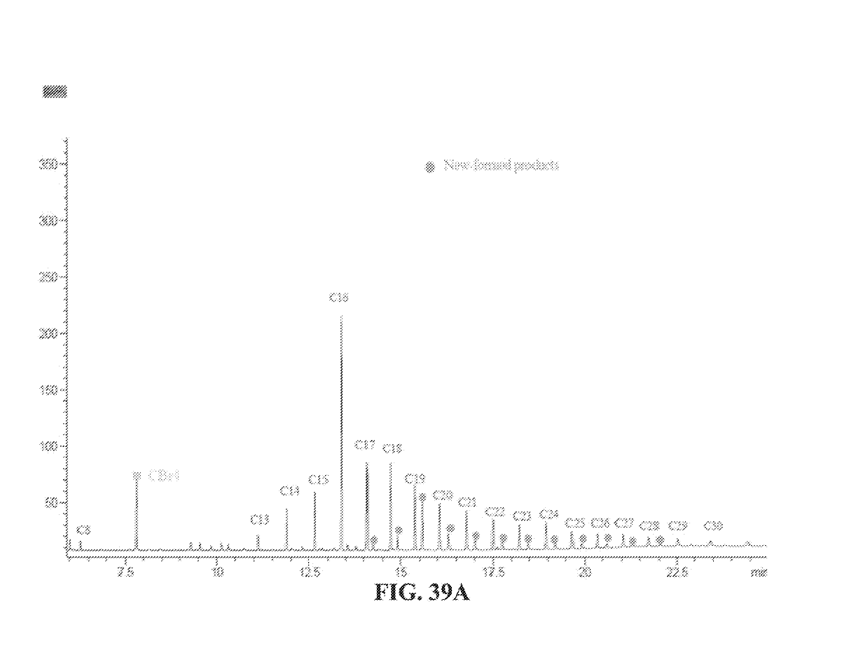

Metathesis of cyclodecane gave also similar distribution of lower and higher cyclic alkanes (FIGS. 36-37). In this case, the ring contraction products are cyclooctane, cycloheptane, cyclohexane and cyclopentane. A distribution of macrocyclic alkanes is also observed from cyclododecane (cC₁₂) to cyclotetracontane (cC₄₀). It should be noted that formation of cyclononane from contraction of cyclodecane was not observed. In the same reaction conditions, no metathesis products were observed when cyclopentane, cyclohexane and cycloheptane were used as substrate.

Metathesis reaction of cyclooctane or cyclodecane catalyzed by species 2 produces a distribution of higher and lower cyclic alkanes. On the basis of the seminal work on light alkane metathesis, the multifunctional precursor catalyst for this transformation operates as follow: i) C—H bond activation, ii) alpha or beta-H elimination to give W-carbene hydride and an olefin, iii) intermolecular reaction of this in situ formed olefin with the carbene, which after cycloreversion [2+2] of the metallacycle gives a new carbene and a new olefin and finally two different hydrocarbons via iv) stepwise hydrogenation of double bond. Thus, for cyclooctane metathesis, a C—H activation followed by beta-H elimination should lead to the dehydrogenation of cyclooctane to cyclooctene. See, D. Michos, X. L. Luo, J. W. Faller, R. H. Crabtree, Inorg Chem 1993, 32, 1370, which is incorporated by reference in its entirety. This olefin would undergo successive ring opening-ring closing metathesis reactions (ROM-RCM). Finally, a hydrogenation step of these double bonds gives the corresponding macrocyclic alkanes. Since, the mechanism postulated involves the formation of cyclooctene, not detected at the end of a typical catalytic run, this metathesis was performed in a NMR Young tube in which the hydrogen formed was released continuously over a long period of time. Indeed, after 10 days, ¹³C NMR spectroscopy displays a very weak signal at 130 ppm assigned to cyclooctene (GC and GCMS) (FIG. 37). These results point out that the cyclooctene is effectively formed in situ as an intermediate, which supports our initially proposed mechanism. See, J. M. Basset, C. Coperet, L. Lefort, B. M. Maunders, O. Maury, E. Le Roux, G. Saggio, S. Soignier, D. Soulivong, G. J. Sunley, M. Taoufik, J. Thivolle-Cazat, J Am Chem Soc 2005, 127, 8604, which is incorporated by reference in its entirety.

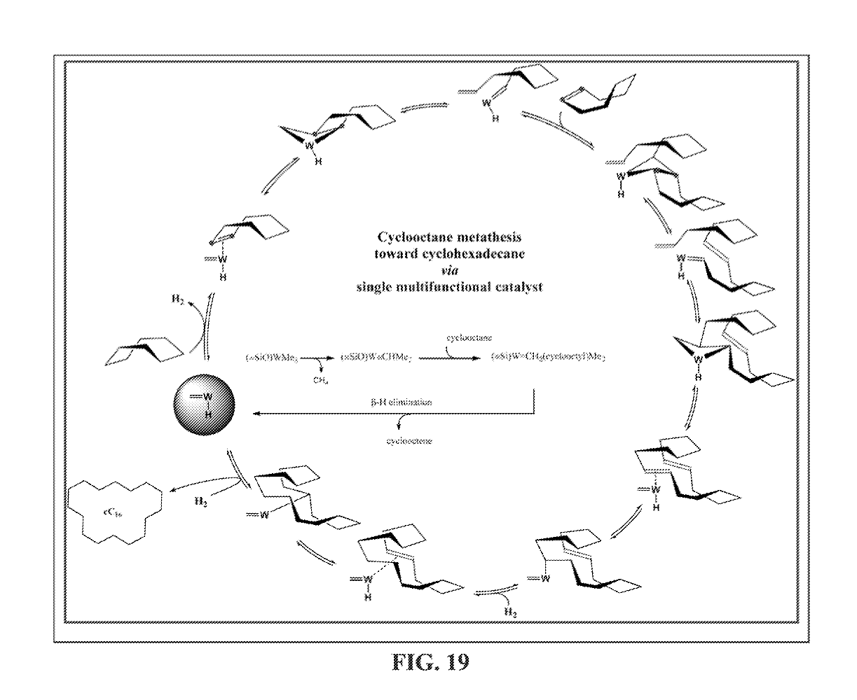

In the cyclooctane metathesis, this cyclooctene intermediate would coordinate to W-methylidene which is generated from species 2 as reported earlier (FIG. 19). The next step would follow a classical ROM-RCM of cyclooctene by backbiting of terminal double bond to produce 1,9-cyclohexadecadiene. Finally, hydrogenation of this macrocyclic diene intermediate would lead to the observed cyclohexadecane. Successive insertions of cyclooctene by ROM and RCM would generate other macrocyclic alkanes with multiple carbon numbers of 8. In this catalytic system, a steady state concentration of minute amounts of coordinated cyclooctene prevents the formation of polymeric products.

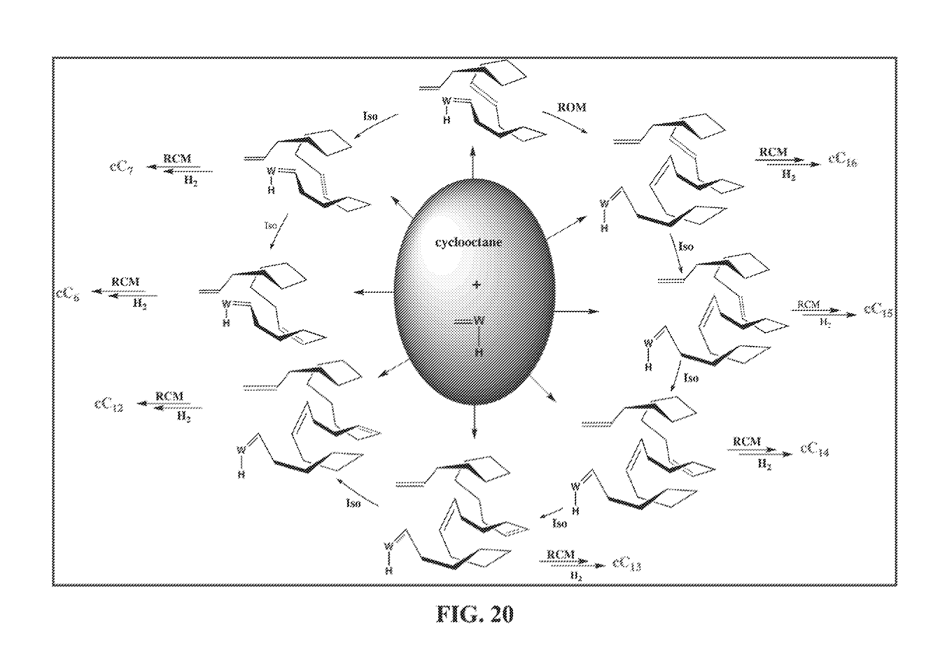

The formation of cyclic alkanes and the other macrocyclic alkanes is resulting from double bond isomerization process prior to RCM. W-hydride is likely responsible for this isomerization step. For instance, starting from C₈ W-alkylidene, an isomerization of the terminal olefin followed by RCM and hydrogenation steps would provide cycloheptane (FIG. 20). Only the formation of some products is depicted. It is an example of how ROM, RCM and isomerisation process could evolve during the reaction, indeed each internal olefin could be isomerized and successive ROM/RCM could occur at any time providing miscellaneous cyclic and macrocyclic-alkanes. For example, isomerisation of the terminal olefin before RCM (backbiting) could also explain the distribution of cyclooctane metathesis reaction products. However, the same process could explain all macrocyclic-alkanes resulting from cyclooctane metathesis reaction: successive ROM and RCM reactions in competition of internal olefins isomerization involving either the carbene or the hydride functions of the propagative species.

High selectivity of cyclohexadecadiene (dimer) is obtained in the earlier hours of this reaction. It has been observed with both supported and unsupported Ru catalysts that selective formation of cyclic dimer requires a kinetic-reaction regime, low temperature and high dilution of cyclooctene to avoid the undesirable polymerization reaction. See, S. Kavitake, M. K. Samantaray. R. Dehn, S. Deuerlein, M. Limbach, J. A. Schachner, E. Jeanneau, C. Coperet, C. Thieuleux, Dalton T 2011, 40, 12443, and M. K. Samantaray, J. Alauzun, D. Gajan, S. Kavitake, A. Mehdi, L. Veyre, M. Lelli, A. Lesage, L. Emsley, C. Coperet, C. Thieuleux, J Am Chem Soc 2013, 135, 3193, each of which is incorporated by reference in its entirety. This multifunctional alkane metathesis allows the use of directly neat cyclooctane without dilution. Moreover, if the reaction is carried out without stirring, the conversion is decreased and one needs 24 hours to reach the conversion obtained within 6 hours (under stirring conditions) with dimer selectivity up to 41%. This result highlights the importance effect of stirring and the mean residence time. See. M. Bru, R. Dehn, J. H. Teles, S. Deuerlein, M. Danz, I. B. Muller, M. Limbach, Chem-Eur J 2013, 19, 11661, and J. Cabrera, R. Padilla, M. Bru, R. Lindner, T. Kageyama, K. Wilckens, S. L. Balof, H. J. Schanz, R. Dehn, J. H. Teles, S. Deuerlein, K. Muller, F. Rominger, M. Limbach, Chem-Eur J 2012, 18, 14717 cS. Warwel, H. Katker, C. Rauenbusch, Angewandte Chemie-International Edition in English 1987, 26, 702, each of which is incorporated by reference in its entirety.

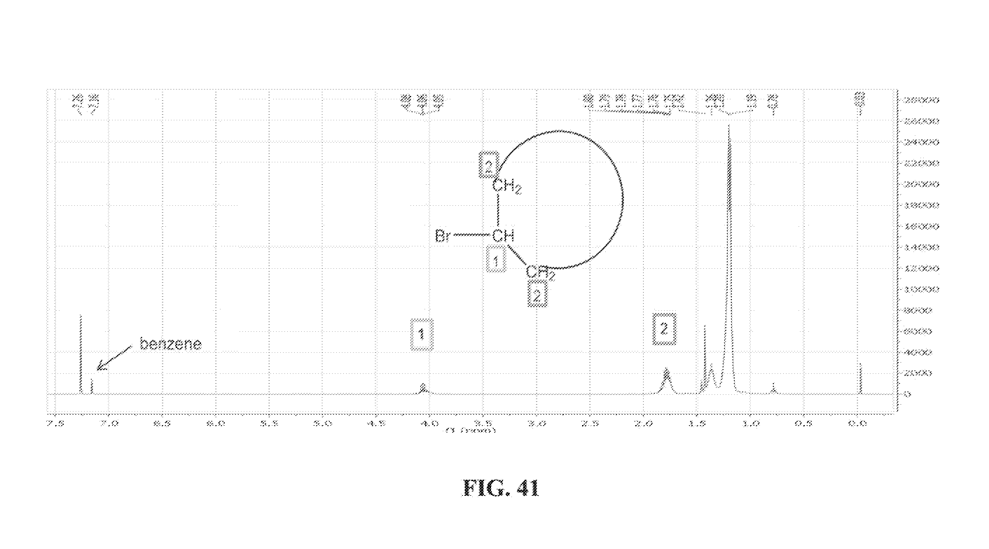

To see whether the formation of observed ring contraction cyclic alkanes could also arise from secondary metathesis of macrocyclic alkanes, the reactivity of a fraction of cC₁₂-cC₄₀ was examined. This colorless oil was easily isolated by removal of cyclic alkanes under reduced pressure (FIG. 41). No ring contraction of cyclic products was observed with 1 after 48 h at 150° C. Thus, this would suggest that the formation of cC₅, cC₆ and cC₇ results directly from the isomerization of C8 W-alkylidene intermediate (FIG. 20) and they accumulate over a long period of time. It is known that ROMP of cyclic olefins depends on the ring strain of the monomer as well as the reaction conditions (e.g. temperature, concentration of monomer, pressure). See, P. V. Schleyer, J. E. Williams, K. R. Blanchard, J Am Chem Soc 1970, 92, 2377, which is incorporated by reference in its entirety. In the present case, there is a competition between ROM/double bond isomerization/RCM leading to cyclic alkanes and ROM/backbiting affording the macrocyclic alkanes.

### Functionalization of Macrocyclic Alkanes

Macrocylic alkanes can be further functionalized (e.g. amidation, bromination) For example, medium-size alkanes, such as cyclooctane or cyclodecane, can be used for bromination based on a radicalary mechanism (scheme 8).

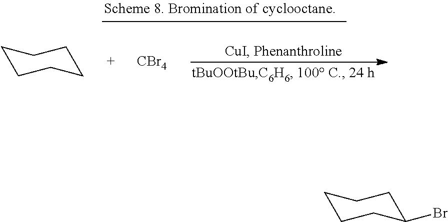 [SMILES: C1CCCCC1 ; "CBr4" ; BrC1CCCCC1]

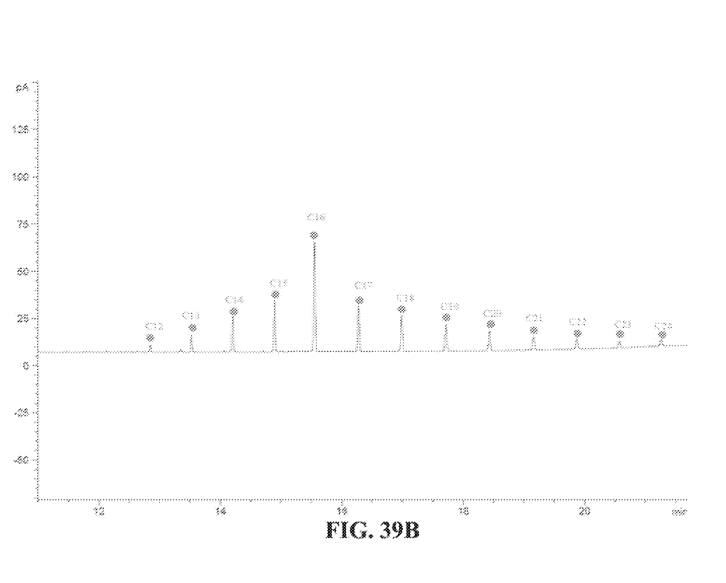

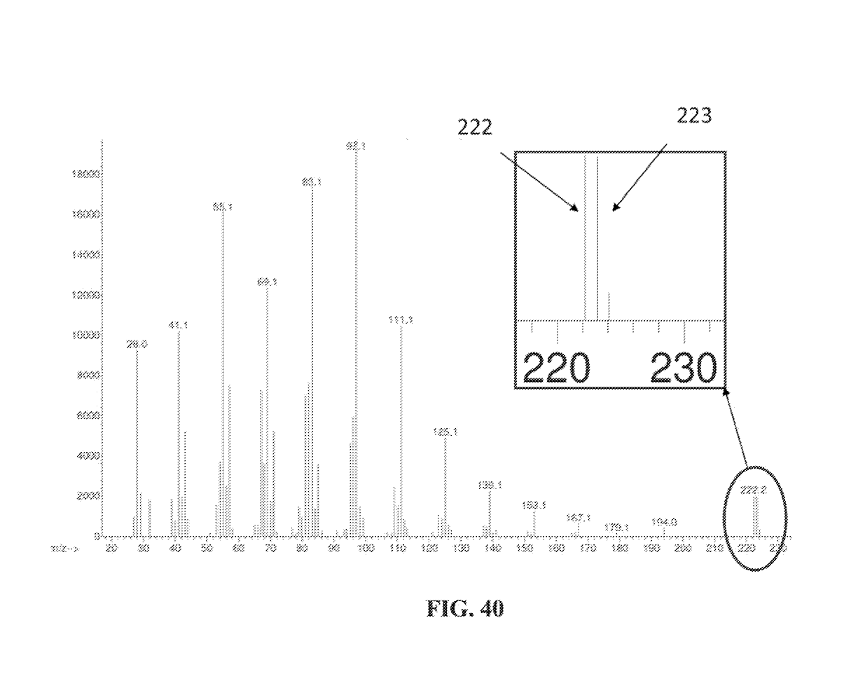

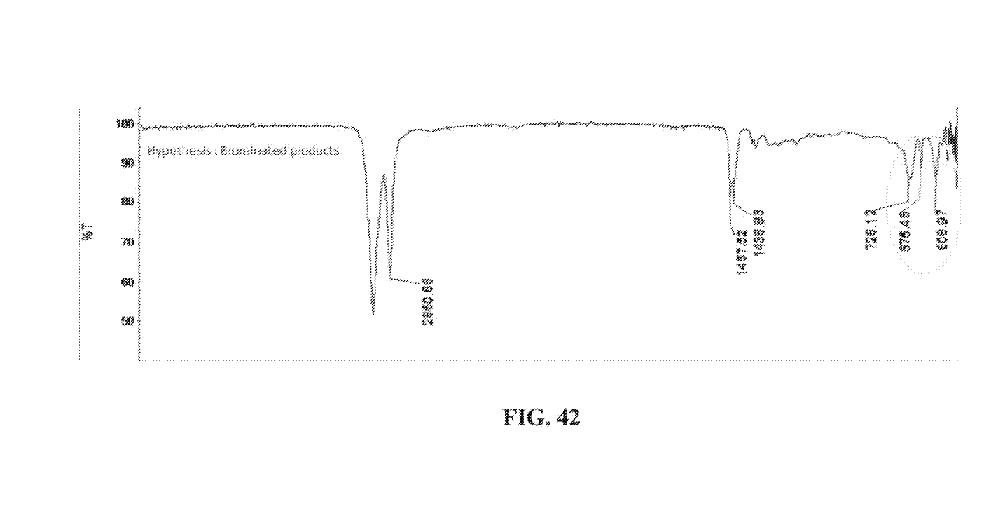

GC chromatogram (FIG. 39A) shows a typically crude reaction mixture from the bromination macrocyclic alkanes, where the green dots show newly-formed brominated products. Since high dilution and excess of cyclic alkanes are required for this transformation, this reaction is not completed. Nonetheless, silica-gel chromatography purification is sufficient for the isolation of pure brominated macrocyclic alkanes (FIG. 39B) Next, analysis by GC-MS reveals ion fragmentations (m/z 222 and 233) with a 1:1 ratio characteristic of the presence of Br atom (FIG. 40). Further NMR spectroscopies show a broad signal proton resonance centered at 4.05 ppm assigned to a —CH—Br bond (FIG. 41). Infra-red spectroscopy displays a C—Br stretch at 609 and 676 cm⁻¹ characteristic of alkyl halides (FIG. 42).

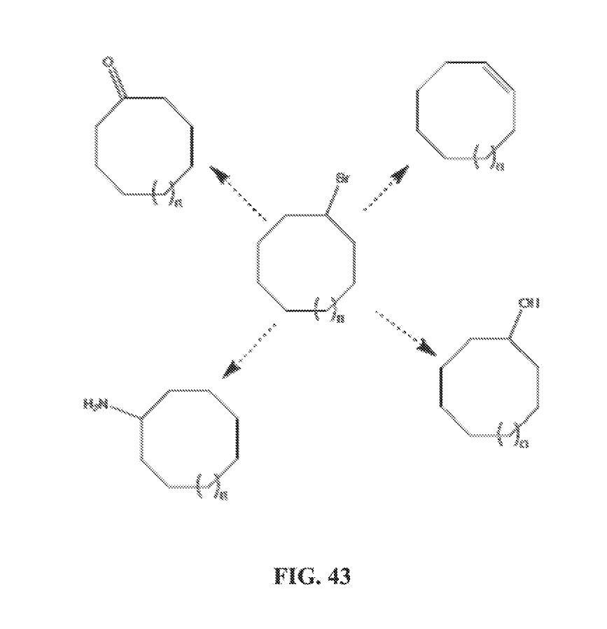

The isolation of these brominated macrocyclic products could serve as building blocks for the production of other functionalized macrocyclic products such as alkenes, ketones, alcohols or amines (FIG. 43)

### General Procedure:

All experiments were carried out by using standard Schlenk and glovebox techniques under an inert nitrogen atmosphere. The syntheses and the treatments of the surface species were carried out using high vacuum lines (<10⁻⁵ mbar) and glove-box techniques. Pentane was distilled from a Na/K alloy under N₂ and dichloromethane from CaH₂. Both solvents were degassed through freeze-pump-thaw cycles. SiO₂₋₇₀₀ and SiO₂₋₂₀₀ were prepared from Aerosil silica from Degussa (specific area of 200 m²/g), which were partly dehydroxylated at either 700° C. or 200° C. under high vacuum (<10⁻⁵ mbar) for 24 h to give a white solid having a specific surface area of 190 m²/g and containing respectively 0.5-0.7 OH/nm² and 2.4-2.6 OH/nm². Hydrogen and propane were dried and deoxygenated before use by passage through a mixture of freshly regenerated molecular sieves (3 Å) and R3-15 catalysts (BASF). IR spectra were recorded on a Nicolet 6700 FT-IR spectrometer by using a DRIFT cell equipped with CaF₂ windows. The IR samples were prepared under argon within a glovebox. Typically, 64 scans were accumulated for each spectrum (resolution 4 cm⁻¹). Elemental analyses were performed at Mikroanalytisches Labor Pascher (Germany). Gas phase analysis of alkanes was performed using an Agilent 6850 gas chromatography column with a split injector coupled with a FID. A HP-PLOT/U 30 m×0.53 mm; 20.00 mm capillary column coated with a stationary phase of divinylbenzene/ethylene glycol dimethylacrylate was used with nitrogen as the carrier gas at 32.1 kPa. Each analysis was carried out with the same conditions: a flow rate of 1.5 mL/min and an isotherm at 80° C.

Cyclic alkanes were purchased from Aldrich, distilled from sodium/potassium alloy under nitrogen, degassed via several freeze-pump-thaw cycles, filtered over activated alumina and stored under nitrogen. Octylidenecyclooctane was synthesized in two steps from cyclooctanone according to W. Giencke, O. Ort, H. Stark, Liebigs Annalen Der Chemie 1989, 671, which is incorporated by reference in its entirety. Supported pre-catalyst [(≡SiO)W(Me)₅] was prepared according to M. K. Samantaray, E. Callens, E. Abou-Hamad, A. J. Rossini, C. M. Widdifield, R. Dey, L. Emsley, J. M. Basset, J Am Chem Soc 2014, 136, 1054, which is incorporated by reference in its entirety.

### Liquid State Nuclear Magnetic Resonance Spectroscopy:

All liquid state NMR spectra were recorded on Bruker Avance 600 MHz spectrometers. All chemical shifts were measured relative to the residual ¹H or ¹³C resonance in the deuteurated solvent: CD₂Cl₂, 5.32 ppm for ¹H, 53.5 ppm for ¹³C.

### Solid State Nuclear Magnetic Resonance Spectroscopy:

One dimensional ¹H MAS, ¹³C CP/MAS and ²⁹Si CP/MAS solid state NMR spectra were recorded on Bruker AVANCE III spectrometers operating at 400 MHz, 500 MHz or 700 MHz resonance frequencies for ¹H. In all cases the samples were packed into rotors under inert atmosphere inside gloveboxes. Dry nitrogen gas was utilized for sample spinning to prevent degradation of the samples. NMR chemical shifts are reported with respect to the external references TMS and adamantane. For ¹³C and ²⁹Si CP/MAS NMR experiments, the following sequence was used: 90° pulse on the proton (pulse length 2.4 s), then a cross-polarization step with a contact time of typically 2 ms, and finally acquisition of the ¹³C and ²⁹Si signal under high power proton decoupling. The delay between the scans was set to 5 s to allow the complete relaxation of the ¹H nuclei and the number of scans ranged between 3 000-5 000 for ¹³C, 30 000-50 000 for ²⁹Si and was 32 for ¹H. An exponential apodization function corresponding to a line broadening of 80 Hz was applied prior to Fourier transformation.

The 2D ¹H-¹³C heteronuclear correlation (HETCOR) solid state NMR spectroscopy experiments were conducted on a Bruker AVANCE III spectrometer using a 3.2 mm MAS probe. The experiments were performed according to the following scheme: 90° proton pulse, t₁ evolution period, CP to ¹³C, and detection of the ¹³C magnetization under TPPM decoupling. For the cross-polarization step, a ramped radio frequency (RF) field centered at 75 kHz was applied to the protons, while the ¹³C channel RF field was matched to obtain optimal signal. A total of 32 t₁ increments with 2000 scans each were collected. The sample spinning frequency was 8.5 kHz. Using a short contact time (0.5 ms) for the CP step, the polarization transfer in the dipolar correlation experiment was verified to be selective for the first coordination sphere about the tungsten, that is to lead to correlations only between pairs of attached ¹H-¹³C spins (C—H directly bonded).

¹H-¹H Multiple-Quantum Spectroscopy

Two-dimensional double-quantum (DQ) and triple-quantum (TQ) experiments were recorded on a Bruker AVANCE III spectrometer operating at 600 MHz with a conventional double resonance 3.2 mm CP/MAS probe, according to the following general scheme: excitation of DQ coherences, t₁ evolution, z-filter, and detection. The spectra were recorded in a rotor synchronized fashion in t₁; that is the t₁ increment was set equal to one rotor period (45.45 μs). One cycle of the standard back-to-back (BABA) recoupling sequences was used for the excitation and reconversion period. See, for example, Sommer, W. et al., J. Magn. Reson. 1995, 113, 131-134, which is incorporated by reference in its entirety. Quadrature detection in w₁ was achieved using the States-TPP1 method. A spinning frequency of 22 KHz was used. The 90° proton pulse length was 2.5 μs, while a recycle delay of 5 s was used. A total of 128 t₁ increments with 32 scans per each increment were recorded. The DQ frequency in the w₁ dimension corresponds to the sum of two single quantum (SQ) frequencies of the two coupled protons and correlates in the w₂ dimension with the two corresponding proton resonances. See, for example, Rataboul, F. et al., J. Am. Chem. Soc. 2004, 126, 12541-12550, which is incorporated by reference in its entirety. The TQ frequency in the w₁ dimension corresponds to the sum of the three SQ frequencies of the three coupled protons and correlates in the w₂ dimension with the three individual proton resonances. Conversely, groups of less than three equivalent spins will not give rise to diagonal signals in the spectrum.

### Preparation of Hexamethyltungsten, WMe₆ 1.

The molecular precursor WMe₆ 1 was prepared from WCl₆ and (CH₃)₂Zn, following the literature procedure. See, for example, Shortland, A. J. et al., Science 1996, 272, 182-183, which is incorporated by reference in its entirety. To a mixture of WCl₆ (1.80 g, 4.5 mmol) in dichloromethane (25 mL), was added Zn(CH₃)₂ (13.6 mmol, 1.0 M in heptane) at −80° C., and after addition, the reaction mixture was allowed to warm to −35° C. and stirred at this temperature for another 30 minutes. After successive filtrations with pentane and removal of the solvent, a red solid 1 was obtained (0.16 g, 12%). [caution: this 12 e⁻ compound is highly unstable and is prone to violent decomposition]. See, for example, Seppelt, K. Science 1996, 272, 182-183, which is incorporated by reference in its entirety. ¹H NMR (CD₂Cl₂, 600 MHz): δ (ppm)=1.65 (s, 18H, WCH₃). ¹³C NMR (CD₂Cl₂, 150 MHz): δ (ppm)=82 (s, 6H, J₁₈₃_{_{W}}_{.13}_{_{C}}=47 Hz, WCH₃). HSQC confirms the correlation between the ¹H and ¹³C NMR signals.

The ¹³C enriched W(CH₃)₆ was synthesized as described below: ¹³C enriched (¹³CH)₂Zn was prepared from a suspension of ¹³CH₃Li and ZnCl₂ (2:1) with subsequent synthetic steps being analogous to those provided above. See, for example, DuMez, D. D. et al., J. Am. Chem. Soc. 1996, 118, 12416-12423, which is incorporated by reference in its entirety.

Preparation of WMe₆ on SiO₂₋₇₀₀ 2

A solution of 1 in pentane (150 mg, 1.2 equivalents with respect to the amount of surface accessible silanols) was reacted with 1.8 g of AEROSIL SiO₂₋₇₀₀ at −50° C. for one hour, was allowed to warm to −30° C., and was stirred for an additional 2 hours. At the end of the reaction, the resulting yellow solid was washed with pentane (3×20 mL) and dried under dynamic vacuum (<10⁻⁵ Torr, 1 h). IR data (cm⁻¹): 3742, 3014, 2981, 2946, 2878, 1410. ¹H solid-state NMR (400 MHz): δ (ppm)=2.0 (W—CH₃). ¹³C CP/MAS solid-state NMR (100 MHz): δ (ppm)=82.0 (W—CH₃). Elemental analysis: W: 3.5-3.9% wt, C: 1.1-1.3% wt. C/W ratio obtained 5.0+/−0.1 (expected was 5).

### Preparation of 3

The same procedure above with Aerosil SiO₂₋₂₀₀ dehydroxylated at 200° C. ¹H solid-state NMR (400 MHz): δ (ppm)=2.0 (W—CH₃). ¹³C CP/MAS solid-state NMR (100 MHz): δ (ppm)=82.0 (W—CH³). Elemental analysis: W: 3.49% wt, C: 1.04% wt. C/W ratio obtained 4.6+/−0.1 (expected, 4.7).

### Synthesis of 5 and 6

In a glass reactor, 1.25 g of 2 was added and heated at 100° C. (ramped at 60° C./h) for 12 hours to produce a grey/dark colored powder which is a mixture of the monopodal and bipodal species, 5 and 6. IR data (cm⁻¹): 3741, 2967, 2929, 2899. ¹H solid-state NMR (400 MHz): δ (ppm)=−0.5 (s, Si—CH₃), 1.1 (s, W—CH₃), 1.4 (s, W—CH₃), 2.0 (s, W—CH₃), 4.1 (s, W—CH₃), 7.6 (s, W≡CH). ¹³C CP/MAS solid-state NMR (100 MHz): δ (ppm)=40 (s, W—CH₃), 44 (s, W—CH₃), 48 (s, W≡CH), 298 (s, W≡CH). ²⁹Si CP/MAS solid-state NMR (80 MHz): δ (ppm)=−12.2 (≡SiCH₃); −100 (Q3), −108 (Q4). Elemental analysis: W: 3.18% wt. C: 0.6% wt. C/W ratio obtained 2.9+/−0.1 (expected, 3).

### Synthesis of Octylcyclooctane

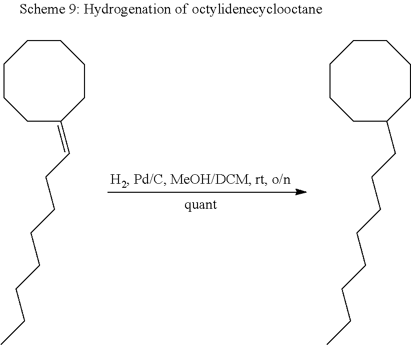

Freshly distilled methanol (10 mL) and dichloromethane (1 mL) were introduced to a flask containing octylidenecyclooctane (370 mg, 1.66 mmol). Next, Pd/C (80 mg) was added to the solution previously purged with nitrogen. The reaction mixture was treated under 1 atm of H₂ at room temperature overnight. After filtration through celite and concentration under reduced pressure, the resulting oil was purified over silica column chromatography (pentane as eluent) to yield octylcyclooctane as colorless oil (350 mg, 94%). ¹H NMR δ_{H} (CDCl₃, 600 MHz) 1.66-1.55 (m, 7H, —CH₂—), 1.48-1.40 (m, 6H, —CH₂—), 1.32-1.20 (m, 14H, —CH₂), 1.18-1.15 (m, 2H, —CHCH₂—), 0.88 (tp, 3H, J=6.9 Hz, CH₂CH₃). ¹³C NMR δ_{C} (CDCl₃, 125 MHz) 38.5 (CH₂), 37.8 (CH), 32.7 (2CH₂×2), 32.1 (CH₂), 30.2 (CH₂), 29.9 (CH₂), 29.5 (CH₂), 27.6 (CH₂), 27.5 (CH₂×2), 26.5 (CH₂), 25.8 (CH₂×2), 22.8 (CH₂), 14.3 (CH₃). MS (EI) m/z 224. Anal. calc. for C₁₆H₃₂: C, 85.63; H, 14.37%. Found: C, 85.65; H, 14.55%.

### Linear Alkanes and Alkylcyclobexanes Formation:

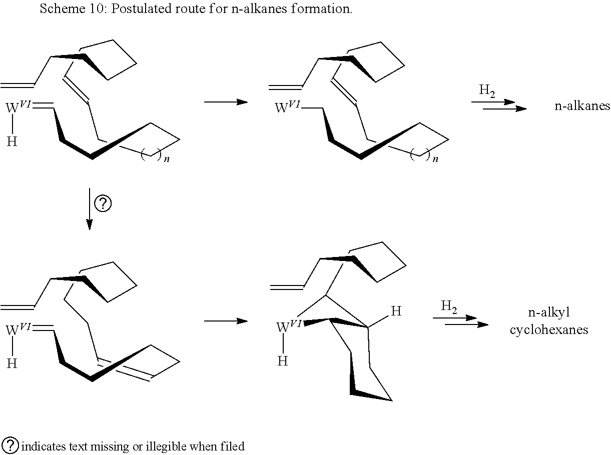

Traces of linear alkanes and alkylcyclohexanes observed could be explained by the reduction of W-alkylidene^{(VI)} to W^{(IV)}, followed by a stepwise hydrogenolysis with H₂. See, Wang, S. Y. S.; VanderLende, D. D.; Abboud, K. A.; Boncella, J. M. Organometallics 1998, 17, 2628, and Merle, N.; Stoffelbach, F.; Taoufik, M.; Le Roux, E.; Thivolle-Cazat, J.; Basset, J. M. Chem Commun 2009, 2523, each of which is incorporated by reference in its entirety. These products could account for the deactivation of this supported catalyst.

### Procedure for the Quantification of Methane Released During Hydrogenolysis:

A sample of 2 (0.020 mmol/W, 100 mg) and dry H₂ (786 hPa) was added in a batch reactor of known volume (480 mL). The reaction mixture was heated to 130° C. for 10 hours. Next, an aliquot of the gas phase released was analyzed by GC. Gas phase analysis gave 0.098 mmol of CH₄, corresponding to a C/W ratio of 4.9+/−0.1 (expected, 5).

### Typical Procedure for Propane Metathesis Reactions:

A mixture of a potential catalytic material (0.013 mmol/l W) and dry propane (980-1013 hPa) were heated to 150° C. in a batch reactor of known volume (480 mL) over a 5 day period. At the end of the run, an aliquot was drawn and analyzed by GC. The selectivities are defined as the amount of products over the total amount of products.

### General Procedure for Cyclic Alkanes Metathesis Catalytic Runs:

All the reactions were carried out following the same way: an ampoule is filled with the catalyst (50 mg, 6.5 μmol, W loading: 2.4% wt, 0.2% equivalent) in a glove box and the cyclic alkane (0.5 mL, 3.7 mmol) is then added. The ampoule is sealed under vacuum, immersed in an oil bath and heated at 150° C. At the end of the reaction, the ampoule is allowed to cool to −78° C. Then, the mixture is diluted by addition of external standard n-pentane and after filtration the resulting solution is analysed by GC and GC/MS. For kinetic studies, each analysis represents an independent run.

### Catalytic Cyclooctane Metathesis Using NMR Young Tube:

A Young NMR tube (equipped with external deuterated toluene) was charged with 1 (50 mg, 6.5 μmol, W loading: 2.4% wt, 0.2% equivalent) in a glove box and cyclooctane (0.5 mL, 3.7 mmol) is then added. The NMR tube is inserted in an oil bath and heated at 150° C. Periodically, the NMR tube is removed from the bath, allowed to cool to room temperature and analysed by 13C NMR. At the end of the reaction, the mixture is diluted by addition of external standard n-pentane and after filtration, the resulting solution is analysed by GC and GC/MS.

### Gas Chromatography (GC):

GC measurements were performed with an Agilent 7890A Series (FID detection). Method for GC analyses: Column HP-5: 30 m length×0.32 mm ID×0.25 μm film thickness; Flow rate: 1 mL/min (N2); split ratio: 50/1; Inlet temperature: 250° C., Detector temperature: 250° C.; Temperature program: 40° C. (1 min), 40-250° C. (15° C./min), 250° C. (1 min), 250-300° C. (10° C./min), 300° C. (30 min); Cyclic alkanes retention time: tR (cyclooctane): 6.51 min, tR (cyclohexadecane, dimer): 13.56 min, tR (cyclotetraeicosane, trimer): 19.30 min.

### GC-MS (Mass Spectroscopy):

GC-MS measurements were performed with an Agilent 7890A Series coupled with Agilent 5975C Series. GC/MS equipped with capillary column coated with none polar stationary phase HP-SMS was used for molecular weight determination and identification that allowed the separation of hydrocarbons according to their boiling points differences. GC response factors of available cC₅-cC₁₂ standards were calculated as an average of three independent runs. The plots of response factor versus cyclic alkanes carbon number were determined and a linear correlation was found. Then, we extrapolated the response factors of this plot for the other cyclic alkanes (FIGS. 21 and 22).

### Ring Opening Metathesis Polymerization (ROMP):

ROMP of cyclooctene catalyzed by species 2. A flame dried ampoule is filled with catalyst 2. (50 mg, 6.5 μmol, W loading: 2.4% wt. 0.2% equivalent) in a glove box and cyclooctene (0.5 mL, 3.7 mmol) is then added. The ampoule is then sealed under vacuum, immersed in an oil bath and heated at 150° C. At the end of the reaction, the ampoule is allowed to cool to −78° C.

Other embodiments are within the scope of the following claims.

1. A catalyst comprising an oxide support and a supported metal alkyl species bound to the oxide support, wherein the supported metal alkyl species is a group V, VI or a group VII metal in its highest oxidation state and the alkyl group is a C1-C4 alkyl.

2. The catalyst of claim 1, wherein the oxide support includes an oxide of silicon, an oxide of titanium, an oxide of aluminum, a mixed silica-alumina, or an aminated oxide of silicon.

3. The catalyst of claim 1, wherein the supported metal alkyl species bound to the oxide support includes a moiety having a formula of (≡Si—O)ₓM(R₁)_{y}(R₂)_{z}, wherein ≡Si—O is a surface Si—O group, wherein R₁ is a C1-C4 alkylidene group or a C1-C4 alkylidyne group, wherein each R₂, independently, is a halogen or C1-C4 alkyl group, wherein x is 1, 2 or 3, y is 0 or 1, and z is 1, 2, 3, 4 or 5, and wherein M is a group VI metal, such that x+2y+z is 6 when R₁ is a C1-C4 alkylidene group or each of two R₁ groups is a C1-C4 alkylidene group, and that x+3y+z is 6 when R₁ is a C1-C4 alkylidyne group.

4. The catalyst of claim 3, wherein M is tungsten or molybdenum.

5. (canceled)

6. The catalyst of claim 3, wherein R₁ is methylidyne.

7. The catalyst of claim 3, wherein R₂ is methyl.

8. The catalyst of claim 3, wherein x is 1 and y is 0.

9. The catalyst of claim 3, wherein x is 1 and y is 1.

10. The catalyst of claim 3, wherein x is 2 and y is 1.

11. The catalyst of claim 1, wherein the supported metal alkyl species bound to the oxide support includes a moiety having a formula of (≡Si—O)ₓM(R₁)_{y}(R₂)_{z}, wherein ≡Si—O is a surface Si—O group, wherein R₁ is a C1-C4 alkylidene group or a C1-C4 alkylidyne group, wherein R₂ is a C1-C4 alkyl group, wherein x is 1, 2 or 3, y is 0 or 1, and z is 1, 2, 3, or 4, wherein M is a group V metal, such that x+2y+z is 5 when R₁ is a C1-C4 alkylidene group or each of two R₁ groups is a C1-C4 alkylidene group and x+3y+z is 5 when R₁ is a C1-C4 alkylidyne group.

12. The catalyst of claim 11, wherein M is tantalum or vanadium.

13. (canceled)

14. The catalyst of claim 11, wherein R₁ is methylidyne.

15. The catalyst of claim 11, wherein R₂ is methyl.

16. The catalyst of claim 1, wherein the catalyst includes both a monopodal species and a bipodal species.

17. A method of preparing a catalyst comprising dehydroxylating a first material that includes an oxide in a heated environment and grafting the dehydroxylated first material with a second material that includes a moiety having a formula of MRₓ in an inert atmosphere, wherein M is a group V or a group VI metal in its highest oxidation state, R is a C1-C4 alkyl group, and x is an integer.

18. The method of claim 17, wherein the first material includes an oxide of silicon, an oxide of aluminium, a mixed silica-alumina, or an aminated oxide of silicon.

19. The method of claim 17, wherein M is tungsten, molybdenum, tantalum, or vanadium.

20.-22. (canceled)

23. The method of claim 17, wherein R is methyl.

24. The method of claim 17, where the inert atmosphere includes argon.

25. A method of converting an alkane into higher and lower homologues comprising contacting a lower alkane or higher alkane with a catalyst comprising an oxide support and a supported metal alkyl species bound to the oxide support, wherein the supported metal alkyl species is a group V, VI or VII metal in its highest oxidation state and the alkyl group is a C1-C4 alkyl.

26.-42. (canceled)
